(19) United States
(12) Patent Application Publication
(10) Pub. No.: US 2020/0100928 A1
(43) Pub. Date: Apr. 2, 2020

(54) ANKLE BRACE DEVICES, SYSTEMS, AND METHODS

(71) Applicant: Shock Doctor, Inc., Fountain Valley, CA (US)

(72) Inventors: William Best, Fountain Valley, CA (US); Blaze Best, New York, NY (US); Bastien Jourde, Montreal (CA)

(21) Appl. No.: 16/584,070

(22) Filed: Sep. 26, 2019

Related U.S. Application Data
(60) Provisional application No. 62/737,356, filed on Sep. 27, 2018.

(51) Int. Cl. A61F 5/01

(52) U.S. Cl. CPC A61F 5/0111

(57) ABSTRACT

An ankle brace includes a main body configured to receive a foot of a wearer and an ankle stay coupled to the main body. The ankle stay includes a superior anterior section, an intermediate anterior section, and an inferior anterior section. The superior anterior section is configured to be disposed anteriorly relative to the ankle of the foot of the wearer, and the superior anterior section includes a first convex edge facing in an anterior direction and a first concave edge facing in a posterior direction. The intermediate anterior section is configured to be disposed inferiorly relative to the ankle of the foot of the wearer. The intermediate anterior section includes a second convex edge facing in the posterior direction and a second concave edge facing in the anterior direction.

### CROSS REFERENCE TO RELATED APPLICATION

The present application claims the benefit of and priority to, under 35 U.S.C. § 119(e), U.S. Provisional Application Ser. No. 62/737,356, filed on Sep. 27, 2018, which is hereby incorporated herein by reference in its entirety for all that it teaches and for all purposes.

### TECHNICAL FIELD

The present disclosure relates generally to protective and supportive athletic gear and methods of making the same. The present disclosure relates to devices and methods of supporting a wearer's ankle, such as ankle straps and ankle braces. More particularly, the present disclosure relates to ankle braces having ankle stays for providing medial/lateral ankle support.

### BACKGROUND

Each year, many people, both athletes and non-athletes, suffer ankle injuries. In some cases, athletes wrap their ankles with adhesive tape in an attempt to prevent ankle injuries and/or to support their ankles after an injury has occurred. In many instances, athletes and others use ankle braces to protect and/or to rehabilitate their ankles.

Devices for supporting or stabilizing the foot or ankle of a wearer may be worn by a wearer for everyday use and/or for use when engaging in physical activity. Injuries to the foot or ankle are common and may affect a wearer's physical ability and/or athletic performance. For certain wearers it may be beneficial to use an artificial structure to support a foot or ankle that has been weakened or injured. Certain rigid structures may be worn through the day and/or when engaging in sports to provide structural support, or prevent further injury. Often a physician or healthcare worker may apply a custom fitted support or structure to the outside of wearer's limb to provide weight bearing support to the wearer's limb.

While a variety of ankle braces are known, there is a desire for continued improvement in the performance and comfort of known ankle braces. Certain support structures for the foot or ankle of a wearer, such as straps or braces, are available and may provide certain advantages such as agility, comfort, or weight bearing capabilities. However, certain options may be unsuitable because of particular characteristics. For example, sleeves that are currently available may be flexible or comfortable, but may not provide adequate support. In other instances, a custom fitted device may be costly and/or require extensive customization for a wearer. Additionally, certain devices that provide structural support may be uncomfortable, or unsuited for use in certain sports that require a particular level of agility or movement by the wearer's limbs. There is thus a need for a device or method for supporting a limb or limbs of a wearer that provides suitable weight bearing capability yet is flexible and comfortable enough to be used during sports and is also cost effective and accessible.

### SUMMARY

In a first example, an ankle brace according to the present disclosure includes a main body configured to receive a foot of a wearer and an ankle stay coupled to the main body. The ankle stay includes a superior anterior section, an intermediate anterior section, and an inferior anterior section. The superior anterior section is configured to be disposed anteriorly relative to the ankle of the foot of the wearer, and the superior anterior section includes a first convex edge facing in an anterior direction and a first concave edge facing in a posterior direction. The intermediate anterior section is coupled to the superior anterior section. The intermediate anterior section is configured to be disposed inferiorly relative to the ankle of the foot of the wearer. The intermediate anterior section includes a second convex edge facing in the posterior direction and a second concave edge facing in the anterior direction. The inferior anterior section is coupled to the intermediate anterior section opposite the superior anterior section.

In a second example, the ankle brace of the first example, wherein the ankle stay further includes a superior posterior section configured to be disposed posteriorly relative to the ankle of the foot of the wearer. The superior posterior section includes a third convex edge facing in the posterior direction and a third concave edge facing in the anterior direction.

In a third example, the ankle brace of the first example or the second example, wherein the ankle stay further comprises an inferior posterior section. The inferior posterior section comprises a fourth convex edge facing in the anterior direction and a fourth concave edge facing in the posterior direction.

In a fourth example, the ankle brace of the first example through the third example, wherein the ankle stay further comprises an inferior portion. The inferior portion couples the inferior anterior section to the inferior posterior section.

In a fifth example, the ankle brace of the first example through the fourth example, wherein the inferior portion is configured to be disposed inferiorly relative to the sole of the foot of the wearer.

In a sixth example, the ankle brace of the first example through the fifth example, wherein the ankle stay is asymmetric over a coronal plane bisecting the ankle of the foot of the wearer.

In a seventh example, the ankle brace of the first example through the sixth example, wherein the inferior anterior section comprises a third convex edge facing in the anterior direction and a third concave edge facing in the posterior direction.

In an eighth example, the ankle brace of the first example through the seventh example, wherein the main body comprises a medial side configured to be disposed on the medial side of the foot of the wearer and a lateral side configured to be disposed on the lateral side of the foot of the wearer, and the ankle stay is disposed at the medial side of the main body.

In a ninth example, the ankle brace of the first example through the eighth example, wherein the main body comprises a medial side configured to be disposed on the medial side of the foot of the wearer and a lateral side configured to be disposed on the lateral side of the foot of the wearer, and the ankle stay is disposed at the lateral side of the main body.

In a tenth example, the ankle brace of the first example through the ninth example, wherein the ankle stay is a lateral ankle stay, and further comprising a medial ankle stay coupled to the main body and disposed at the medial side of the main body.

In an eleventh example, an ankle brace according to the present disclosure includes a main body configured to receive a foot of a wearer and an ankle stay coupled to the main body. The ankle stay comprises an anterior portion, an inferior portion, and a posterior portion. The anterior portion is configured to be disposed anteriorly relative to the ankle of the foot of the wearer. The inferior portion is coupled to the anterior portion and configured to be disposed inferiorly relative to the sole of the foot of the wearer. The posterior portion is coupled to the inferior portion opposite the anterior portion. The posterior portion is configured to be disposed posteriorly relative to the ankle of the foot of the wearer.

In a twelfth example, the ankle brace of the eleventh example, wherein the anterior portion comprises a convex edge facing in an anterior direction and a concave edge facing in a posterior direction.

In a thirteenth example, the ankle brace of the eleventh example or the twelfth example, wherein the anterior portion comprises a concave edge facing in an anterior direction and a convex edge facing in a posterior direction.

In a fourteenth example, the ankle brace of the eleventh example through the thirteenth example, wherein the posterior portion comprises a convex edge facing in an anterior direction and a concave edge facing in a posterior direction.

In a fifteenth example, the ankle brace of the eleventh example through the fourteenth example, wherein the posterior portion comprises a concave edge facing in an anterior direction and a convex edge facing in a posterior direction.

In a sixteenth example, the ankle brace of the eleventh example through the fifteenth example, wherein the main body comprises a medial side configured to be disposed on the medial side of the foot of the wearer and a lateral side configured to be disposed on the lateral side of the foot of the wearer, and the ankle stay is disposed at the medial side of the main body.

In a seventeenth example, the ankle brace of the eleventh example through the sixteenth example, wherein the main body comprises a medial side configured to be disposed on the medial side of the foot of the wearer and a lateral side configured to be disposed on the lateral side of the foot of the wearer, and the ankle stay is disposed at the lateral side of the main body.

In an eighteenth example, the ankle brace of the eleventh example through the seventeenth example, wherein the ankle stay is a lateral ankle stay, and further comprising a medial ankle stay coupled to the main body and disposed at the medial side of the main body.

In a nineteenth example, an ankle brace according to the present disclosure includes a main body configured to receive a foot of a wearer. The main body has a top portion, a bottom portion, a front portion, a rear portion, a first side portion, and a second side portion. The main body further includes a first portion and a second portion. The first portion is disposed between the bottom portion and the top portion and configured to overlay the dorsal surface of the foot of the wearer. The first portion has a first stretchability. The second portion is disposed at the first side portion and the second side portion. The second portion has a second stretchability, and the second stretchability is less than the first stretchability.

In a twentieth example, the ankle brace of the nineteenth example, wherein the second portion is further disposed at the bottom portion of the main body.

In a twenty first example, the ankle brace of the nineteenth example or the twentieth example, further comprising an ankle stay disposed at one of the first side portion and the second side portion.

In a twenty second example, the ankle brace of the nineteenth example or the twentieth example, further comprising a first ankle stay disposed at the first side portion and a second ankle stay disposed at the second side portion.

In a twenty third example, an ankle brace includes a main body configured to receive a foot of a wearer. The main body has a first side portion, a second side portion opposite the first side portion, and a sole configured to underlie the foot of the wearer. The ankle brace further includes a first side stirrup strap coupled to the sole. The first side stirrup strap extends from the sole upwardly and along the first side portion, and the first side stirrup strap detachably couples to the first side portion. The ankle brace further includes a second side stirrup strap coupled to the sole. The second side stirrup strap extends from the sole upwardly and along the second side portion, and the second side stirrup strap detachably couples to the second side portion.

In a twenty fourth example, the ankle brace of the twenty third example, wherein the first side stirrup strap and the second side stirrup strap are monolithically formed with each other.

In a twenty fifth example, an ankle brace includes a main body configured to receive a foot of a wearer. The main body has a sole configured to underlie the foot of the wearer, a top portion configured to overlie the dorsal surface of the foot of the wearer, and a rear portion configured to be disposed posteriorly relative to the foot of the wearer. The ankle brace further includes a first crossing strap and a second crossing strap coupled to the sole. The first crossing strap and the second crossing strap extend in opposite directions from the sole, extend superiorly and cross each other at the top portion, extend posteriorly and crossing each other again at the rear portion, and extend anteriorly such that the second crossing strap overlies the first crossing strap.

I wherein the first crossing strap and the second crossing strap are monolithically formed with each other.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A is a side perspective view of an ankle brace according to an embodiment of the present disclosure being worn on a foot of a wearer.

FIG. 1B is a side perspective view of the ankle brace of FIG. 1A during dorsiflexion of the foot of the wearer.

FIG. 2A is a side view of an ankle stay of the ankle brace of FIG. 1A shown in a flat configuration.

FIG. 2B is a side perspective view of first and second ankle stays of the ankle brace of FIG. 1A shown positioned relative to the foot of the wearer.

FIG. 2C is a front perspective view of the first and second ankle stays of FIG. 2B shown positioned relative to the foot of the wearer.

FIG. 3A is a side perspective view of an ankle brace according to another embodiment of the present disclosure being worn on a foot of a wearer.

FIG. 3B is a side perspective view of the ankle brace of FIG. 3A during dorsiflexion of the foot of the wearer.

FIG. 4A is a side perspective view of an ankle brace according to another embodiment of the present disclosure being worn on a foot of a wearer.

FIG. 4B is a side perspective view of the ankle brace of FIG. 4A during dorsiflexion of the foot of the wearer.

FIG. 5A is a side view of an ankle stay of the ankle brace of FIG. 4A shown in a flat configuration.

FIG. 5B is a side perspective view of the ankle stay of the ankle brace of FIG. 4A shown positioned relative to the foot of the wearer.

FIG. 5C is a front perspective view of ankle stay of the ankle brace of FIG. 4A shown positioned relative to the foot of the wearer

FIG. 6 is a side perspective view of an ankle stay according to another embodiment of the present disclosure shown in a flat configuration.

FIG. 7 is a side perspective view of an ankle stay according to yet another embodiment of the present disclosure shown in a flat configuration.

FIG. 8A is a side perspective view of an ankle brace according to another embodiment of the present disclosure being worn on the foot of the wearer.

FIG. 8B is a front perspective view of the ankle brace of FIG. 8A being worn on the foot of the wearer.

FIG. 8C is a side perspective view of the ankle brace of FIG. 8A being secured to the foot of the wearer.

FIG. 9A is a side perspective view of an ankle brace according to another embodiment of the present disclosure being worn on the foot of the wearer.

FIG. 9B is a front perspective view of the ankle brace of FIG. 9A being worn on the foot of the wearer.

FIG. 9C is a side perspective view of the ankle brace of FIG. 9A being secured to the foot of the wearer by securing an upper rear strap and a lower rear strap.

FIG. 9D is a top perspective view of the ankle brace of FIG. 9A being secured to the foot of the wearer by securing a first side stirrup strap and a second side stirrup strap.

FIG. 10A is a side perspective view of an ankle brace according to another embodiment of the present disclosure being worn on the foot of the wearer.

FIG. 10B is a front perspective view of the ankle brace of FIG. 10A being worn on the foot of the wearer.

FIG. 10C is a side perspective view of the ankle brace of FIG. 10A being secured to the foot of the wearer by securing an upper rear strap and a lower rear strap.

FIG. 10D is a top perspective view of the ankle brace of FIG. 10A being secured to the foot of the wearer by securing a first side stirrup strap and a second side stirrup strap.

FIG. 10E is a side perspective view of the ankle brace of FIG. 10A being secured to the foot of the wearer by securing a first upper wrapping strap and a second upper wrapping strap.

FIG. 11A is a side perspective view of an ankle brace according to another embodiment of the present disclosure being worn on the foot of the wearer.

FIG. 11B is a front perspective view of the ankle brace of FIG. 11A being worn on the foot of the wearer.

FIG. 11C is a side perspective view of the ankle brace of FIG. 11A being secured to the foot of the wearer by securing an upper rear strap and a lower rear strap.

FIG. 11D is a top perspective view of the ankle brace of FIG. 11A being secured to the foot of the wearer by securing a first side stirrup strap and a second side stirrup strap.

FIG. 11E is a side perspective view of the ankle brace of FIG. 11A being secured to the foot of the wearer by securing a first crossing strap.

FIG. 11F is a side perspective view of the ankle brace of FIG. 11A being secured to the foot of the wearer by securing a second crossing strap.

FIG. 12A is a side perspective view of an ankle brace according to another embodiment of the present disclosure being worn on the foot of the wearer.

FIG. 12B is a front perspective view of the ankle brace of FIG. 12A being worn on the foot of the wearer.

FIG. 13A is a side perspective view of an ankle brace according to another embodiment of the present disclosure being worn on the foot of the wearer.

FIG. 13B is a front perspective view of the ankle brace of FIG. 13A being secured to the foot of the wearer.

FIG. 13C is a side perspective view of the ankle brace of FIG. 13A being secured to the foot of the wearer by securing a first rear strap and a second rear strap.

FIG. 13D is a top perspective view of the of the ankle brace of FIG. 13A being secured to the foot of the wearer by positioning a first crossing stirrup strap and a second crossing stirrup strap about a main body of the ankle brace.

FIG. 13E is a side perspective view of the of the ankle brace of FIG. 13A being secured to the foot of the wearer by securing the first crossing stirrup strap.

FIG. 13F is a side perspective view of the of the ankle brace of FIG. 13A being secured to the foot of the wearer by securing the second crossing stirrup strap.

FIG. 13G is a side perspective view of the of the ankle brace of FIG. 13A being secured to the foot of the wearer by securing an upper wrapping strap.

FIG. 14A is a side perspective view of an ankle brace according to another embodiment of the present disclosure being worn on a foot of a wearer.

FIG. 14B is a side perspective view of the ankle brace of FIG. 14A during dorsiflexion of the foot of the wearer.

FIG. 15A is a side view of an ankle stay of the ankle brace of FIG. 14A shown in a flat configuration.

FIG. 15B is a side perspective view of the ankle stay of the ankle brace of FIG. 14A shown positioned relative to the foot of the wearer.

FIG. 15C is a front perspective view of ankle stay of the ankle brace of FIG. 14A shown positioned relative to the foot of the wearer.

FIG. 16 is a side perspective view of an ankle stay according to another embodiment of the present disclosure shown in a flat configuration.

It should be understood that the drawings are intended facilitate understanding of exemplary embodiments of the present invention are not necessarily to scale.

### DETAILED DESCRIPTION

The following description refers to the accompanying drawings which show specific embodiments. Although specific embodiments are shown and described, it is to be understood that additional or alternative features are employed in other embodiments. The following detailed description is not to be taken in a limiting sense, and the scope of the claimed invention is defined by the appended claims and their equivalents.

It should be understood that like reference numerals are intended to identify the same structural components, elements, portions, or surfaces consistently throughout the several drawing figures, as such components, elements, portions, or surfaces may be further described or explained by the entire written specification, of which this detailed description is an integral part. Unless otherwise indicated, the drawings are intended to be read (for example, cross-hatching, arrangement of parts, proportion, degree, etc.) together with the specification, and are to be considered a portion of the written description.

As used herein, “medial” refers generally to a location toward the middle, midline, or median plane of a wearer's body. As used herein, “lateral” refers generally to a location toward the side or outside of a wearer's body. That is the medial side of a wearer's foot is the side that faces inward, generally toward the center of the body and the opposite leg. The lateral side of a wearer's foot is the side that faces outward, generally away from the wearer's body and the opposite foot. Thus the medial side of a wearer's right foot is on the left side of the foot, and the lateral side of a wearer's right foot is on the right side of the foot. The medial side of a wearer's left foot is on the right side of the foot, and the lateral side of a wearer's left foot is on the left side of the foot. As used herein, “anterior” refers generally to a location toward the front of a wearer's body. As used herein, “posterior” refers generally to a location toward the rear of a wearer's body. As used herein, “superior” refers generally to a location toward the top of a wearer's body. As used herein, “inferior” refers generally to a location near the bottom of a wearer's body. As used herein, “sagittal” refers generally to a vertical plane that divides a wearer's body into a left side and a right side, or a vertical plane that divides a wearer's foot into a medial side and transverse side. As used herein, “coronal” refers generally to a vertical plane that divides a wearer's body into an anterior side and a posterior side, or a vertical plane that divides a wearer's foot into an anterior side and a posterior side.

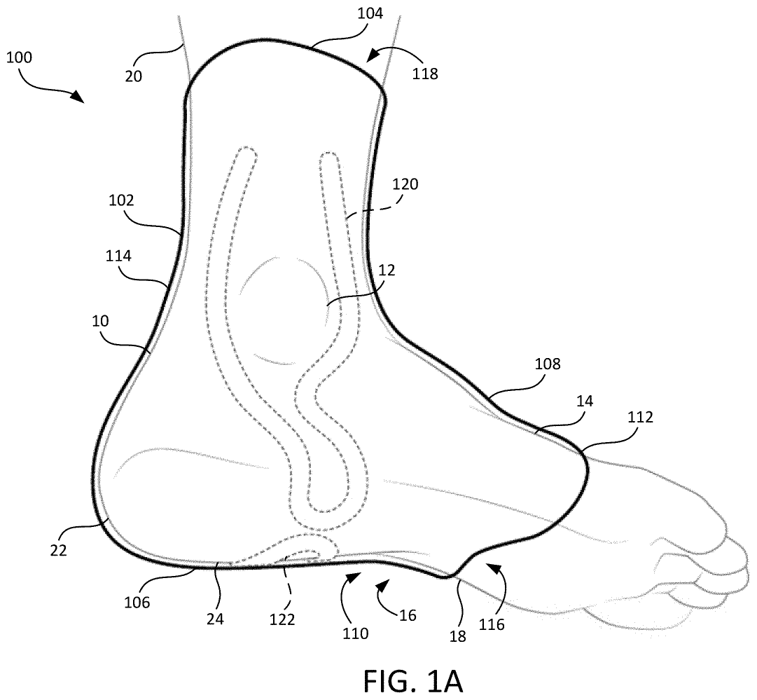

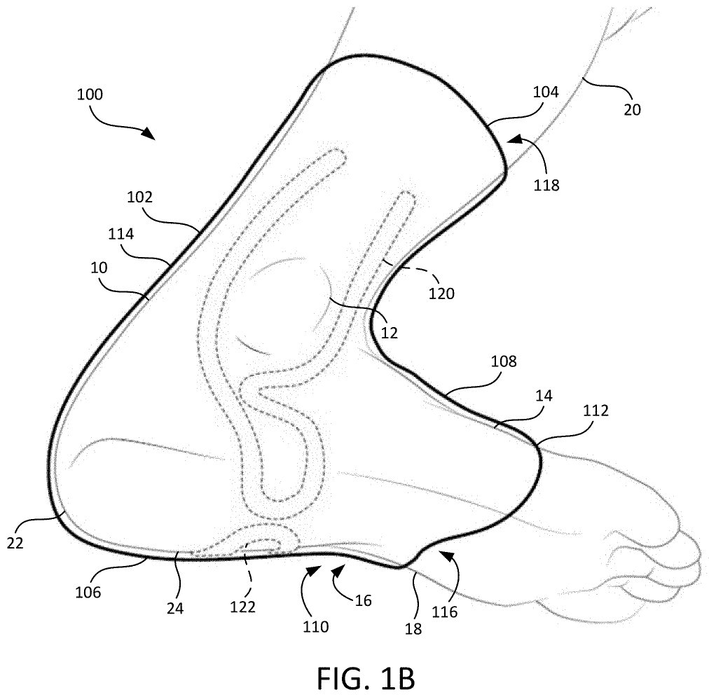

FIGS. 1A and 1B illustrate an ankle brace 100 according to an embodiment of the present disclosure being worn on the foot 10 of a wearer. The ankle brace 100 includes a main body 102 that is configured to receive the foot 10 of the wearer, including the ankle 12. The main body 102 includes a top portion 104, a bottom portion 106, a first side portion 108, a second side portion 110, a front portion 112, and a rear portion 114. In some embodiments, the top portion 104 is located generally on and against the calf 18 of the wearer when worn. In some embodiments, the bottom portion 106 is located generally on and against the sole 24 of the wearer when worn. In some embodiments, the first side portion 108 is located generally on and against the lateral side 14 of the wearer's foot 10 when worn (that is, the first side portion 108 may be a lateral side configured to be disposed on the lateral side 14 of the foot 10 of the wearer). In some embodiments, the second side portion 110 is located generally on and against the medial side 16 of a wearer's foot 10 when worn (that is, the second side portion 110 may be a medial side configured to be disposed on the medial side 16 of the foot 10 of the wearer). In some embodiments, the front portion 112 is located generally on and against the midfoot 20 of the wearer when worn. In some embodiments, the rear portion 114 is located generally on and against the heel 22 and the calf 18 when worn. In some embodiments, the ankle brace 100 is selectively wearable on the right foot or the left foot of the wearer, and/or the ankle brace 100 is symmetric over a sagittal plane bisecting the foot 10 of the wearer.

The front portion 112 of the main body 102 defines a front opening 116 through which the midfoot 18 of the foot 10 extends when worn. The top portion 104 of the main body 102 defines a top opening 118 through which the calf 20 of the wearer extends when worn. In some embodiments, the rear portion 114 of the main body 102 defines a rear opening (not shown) through which the heel 22 of the foot 10 extends when worn. In other embodiments and as shown in the figures, the rear portion 114 of the main body 102 may lack an opening.

The main body 102 may be formed of one or more layers (not shown), each of which may be formed of one or more materials. That is, in some embodiments, the main body 102 is formed of a single layer. In some embodiments, the main body 102 is formed from a plurality of layers. In some embodiments, the plurality of layers of the main body 102 may be coupled to each other via stitching, adhesive, combinations thereof, and the like. In some embodiments, one or more layers of the main body 102 are formed from one or more materials that are resilient yet elastic to support the wearer's foot 10 and allow a wearer to move without restraint. In some embodiments, one or more layers of the main body 102 are formed of fabrics, polymers, composites thereof, and the like. For example, one or more layers of the main body 102 may be formed of neoprene, nylon, polyester, rubber, and latex.

The main body 102 of the ankle brace 100 carries a first ankle support 120, also referred to as a first ankle stay 120, and a second ankle support 122, also referred to as a second ankle stay 122. The ankle stays 120, 122 are configured to partially extend about the ankle 12 of the wearer. In some embodiments, the first ankle stay 120 is disposed at the lateral side 108 of the main body 102 (that is, the first ankle stay 120 may be a lateral ankle stay configured to be disposed on the lateral side 14 of the foot 10 of the wearer). In some embodiments, the second ankle stay 122 is a disposed at the medial side 110 of the main body 102 (that is, the second ankle stay 122 may be a medial ankle stay configured to be disposed on the medial side 16 of the foot 10 of the wearer). In some embodiments, the first ankle stay 120 and the second ankle stay 122 are disposed between layers of the main body 102 and coupled to one or more layers of the main body 102 via adhesives (not shown), stitching (not shown) adjacent to the perimeters of the first ankle stay 120 and the second ankle, or the like.

Generally, the first ankle stay 120 and the second ankle stay 122 provide relatively high resistance to ankle inversion, ankle eversion, and foot plantar flexion. The first ankle stay 120 and the second ankle stay 122 also provide relatively low resistance to foot dorsiflexion (see FIG. 1B). The first ankle stay 120 and the second ankle stay 122 are also configured to be partially disposed below the sole 24 of the foot 10 of the wearer, and the weight of the wearer maintains the first ankle stay 120 and the second ankle stay 122 in positions appropriate for providing resistance to the motions of the foot described above. Shapes and/or other features of the first ankle stay 120 and the second ankle stay 122 that facilitate such characteristics are described in further detail below.

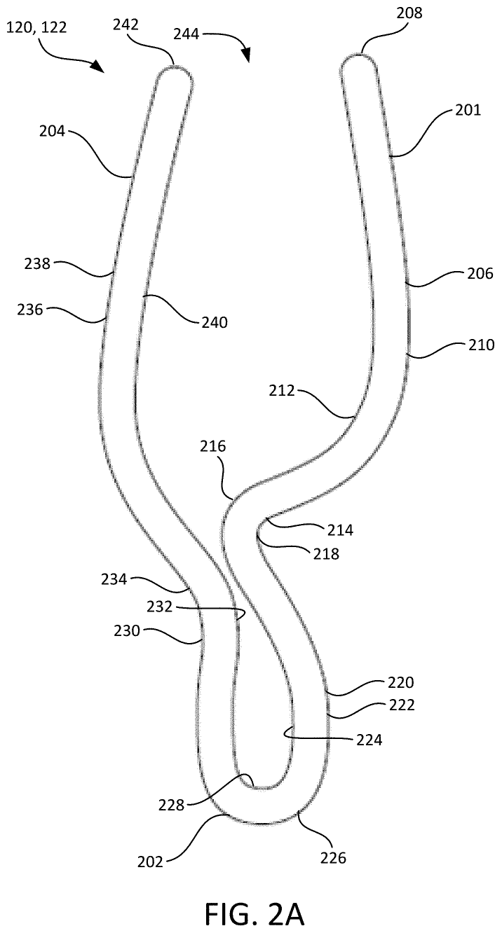

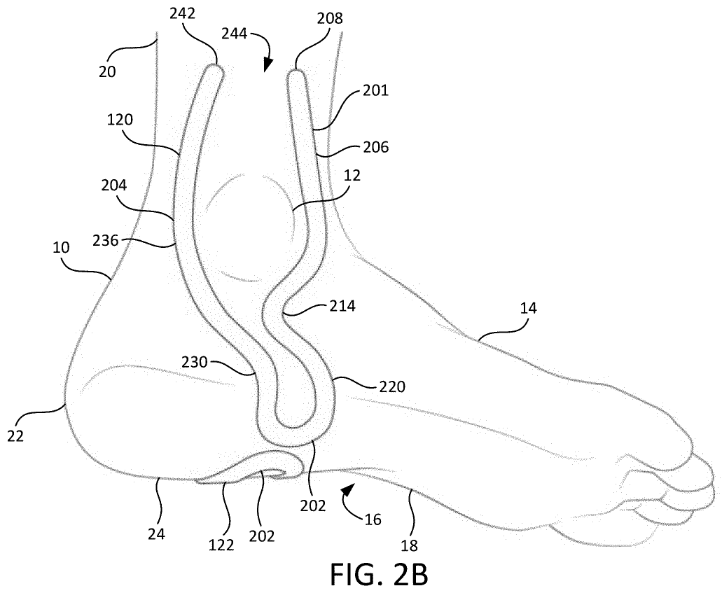

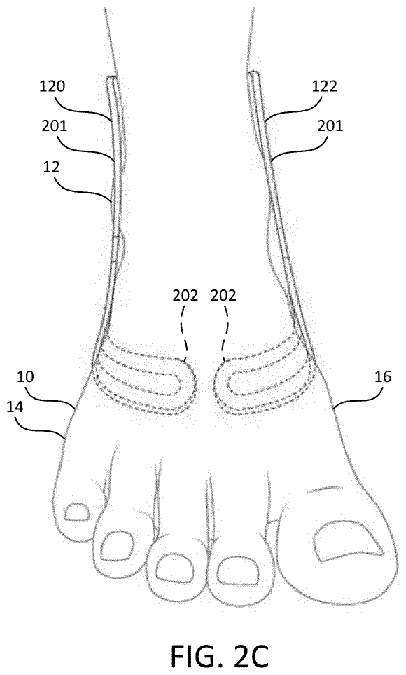

FIG. 2A shows an ankle stay, which may be the first ankle stay 120 and/or the second ankle stay 122, in a flat configuration for illustrative purposes (when carried by the main body 102, the first ankle stay 120 curves into the page and the second ankle stay 122 curves out of the page). FIGS. 2B and 2C illustrate the first ankle stay 120 and the second ankle stay 122 positioned relative to the foot 10 of the wearer, and the main body 102 of the ankle brace 100 is hidden for illustrative purposes.

The ankle stays 120, 122 are monolithic components, although in other embodiments the ankle stays 120, 122 may be formed as separate sections that are joined, for example, via adhesives, ultrasonic welding, or the like. The ankle stays 120, 122 have substantially uniform thicknesses (for example, about 1.5 mm) and widths, although in other embodiments the ankle stays 120, 122 may have non-uniform thicknesses and/or widths. In some embodiments, the ankle stays 120, 122 are formed of polymers (for example, polyethelene), composites (for example, metal reinforced polyethylene), and the like.

The ankle stays 120, 122 are asymmetric over a vertical plane that bisects the ankle 12. The ankle stays 120, 122 include several portions having different shapes that provide the ankle stays 120, 122 with asymmetric shapes. Generally, the ankle stays 120, 122 include an anterior portion 201 that is configured to be disposed anteriorly relative to the ankle 12 of the wearer, an inferior portion 202 that is configured to be disposed inferiorly relative to the sole 24 of the foot 10 of the wearer, and a posterior portion 204 that is configured to be disposed posteriorly relative to the ankle 12 of the foot 10 of the wearer.

The anterior portion 201 includes a superior anterior section 206 that is configured to be disposed anteriorly relative to the ankle 12 of the wearer. The superior anterior section 206 includes an anterior end 208, a first convex edge 210 facing in an anterior direction, and a first concave edge 212 facing in a posterior direction. The first convex edge 210 and the first concave edge 212 provide the superior anterior section 206 with a bend having an angle of about 125 degrees (that is, 125 degrees±5 degrees). In some embodiments, the superior anterior section 206 provides the ankle stays 120, 122 with relatively high resistance to ankle inversion and ankle eversion. Opposite the anterior end 208, the superior anterior section 206 couples to an intermediate anterior section 214.

The intermediate anterior section 214 is configured to be disposed inferiorly relative to the ankle 12 of the wearer. The intermediate anterior section 214 includes a second convex edge 216 facing in the posterior direction and a second concave edge 218 facing in the anterior direction. The second convex edge 216 and the second concave edge 218 provide the intermediate anterior section 214 with a bend having an angle of about 95 degrees (that is, 95 degrees±5 degrees). In some embodiments, the intermediate anterior section 214 provides the ankle stays 120, 122 with relatively low resistance to foot dorsiflexion. Opposite the superior anterior section 206, the intermediate anterior section 214 couples to an inferior anterior section 220.

The inferior anterior section 220 is configured to be disposed to the side of the foot and inferiorly relatively to the sole 24 of the wearer (that is and as shown in FIGS. 2B and 2C, the inferior anterior section 220 curves from the side of the foot 10 of the wearer to below the sole 24 of the wearer). The inferior anterior section 220 includes a third convex edge 222 facing in the anterior direction and a third concave edge 224 facing in the posterior direction. The third convex edge 222 and the third concave edge 224 provide the inferior anterior section 220 with a bend having an angle of about 145 degrees (that is, 145 degrees±5 degrees). In some embodiments, the inferior anterior section 220 provides the ankle stays 120, 122 with relatively high resistance to ankle inversion and ankle eversion and/or is loaded by the weight of the wearer to maintain the ankle stays 120, 122 in appropriate positions as described above. Opposite the intermediate anterior section 214, the inferior anterior section 220 couples to the inferior portion 202.

The inferior portion 202 is configured to be disposed inferiorly relative to the sole 24 of the wearer. The inferior portion 202 includes a fourth convex edge 226 that faces outwardly relative to the anterior portion 201 and the posterior portion 204 of the ankle stays 120, 122. The inferior portion 202 further includes a fourth concave edge 228 that faces inwardly relative to the anterior portion 201 and the posterior portion 204 of the ankle stays 120, 122. The fourth convex edge 226 and the fourth concave edge 228 provide the inferior portion 202 with a general U-shape. In some embodiments, the inferior portion 202 is loaded by the weight of the wearer to maintain the ankle stays 120, 122 in appropriate positions as described above. Opposite the inferior anterior section 220, the inferior portion 202 couples to an inferior posterior section 230 of the posterior portion 204.

The inferior posterior section 230 is configured to be disposed to the side of the foot and inferiorly relatively to the sole 24 of the wearer (that is and as shown in FIGS. 2B and 2C, the inferior anterior section 220 curves from the side of the foot 10 of the wearer to below the sole 24 of the wearer). The inferior posterior section 230 includes a fifth convex edge 232 that faces in the anterior direction and a fifth concave edge 234 that faces in the posterior direction. The fifth convex edge 232 and the fifth concave edge 234 provide the inferior posterior section 230 with a bend having an angle of about 155 degrees (that is, 155 degrees±5 degrees). In some embodiments, the inferior posterior section 230 provides the ankle stays 120, 122 with relatively high resistance to ankle inversion, ankle eversion, and foot plantar flexion and/or is loaded by the weight of the wearer to maintain the ankle stays 120, 122 in appropriate positions as described above. Opposite the inferior portion 202, the inferior posterior section 230 couples to a superior posterior section 236.

The superior posterior section 236 is configured to be disposed posteriorly relative to the ankle 12 of the wearer. The superior posterior section 236 includes a sixth convex edge 238 facing in the posterior direction and a sixth concave edge 240 facing in the anterior direction. The sixth convex edge 238 and the sixth concave edge 240 provide the superior posterior section 236 with a bend having an angle of about 140 degrees (that is, 140 degrees±5 degrees). In some embodiments, the superior posterior section 236 provides the ankle stays 120, 122 with relatively high resistance to ankle inversion, ankle eversion, and foot plantar flexion. Opposite the inferior posterior section 230, the superior posterior section 236 extends to a posterior end 242. The posterior end 242 is disposed posteriorly relative to the anterior end 208 to provide the ankle stays 120, 122 with an open top 244.

Ankle braces according to some embodiments of the present disclosure may include additional or different features, components, shapes, dimensions, and/or characteristics than the ankle brace 100 described above. For example, in some embodiments main bodies of ankle braces include portions having different materials and/or characteristics. An exemplary embodiment of such an ankle brace is described below.

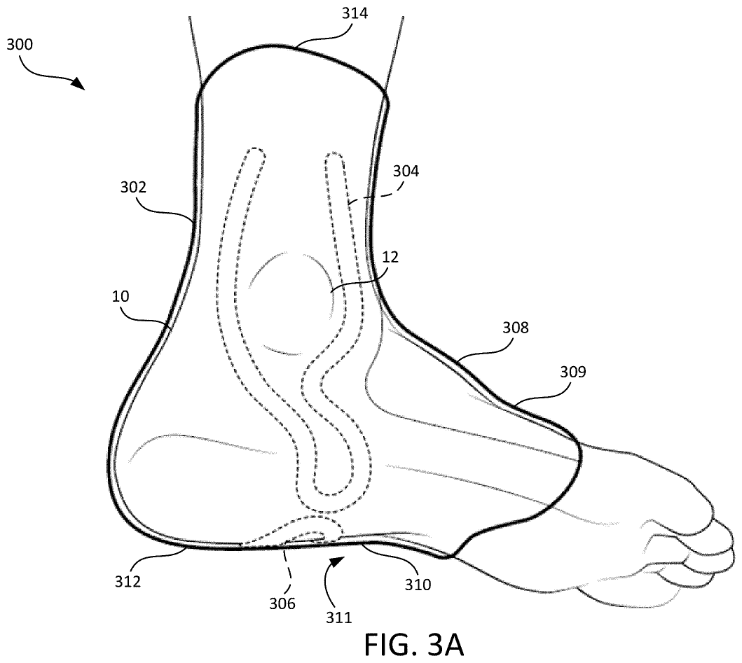

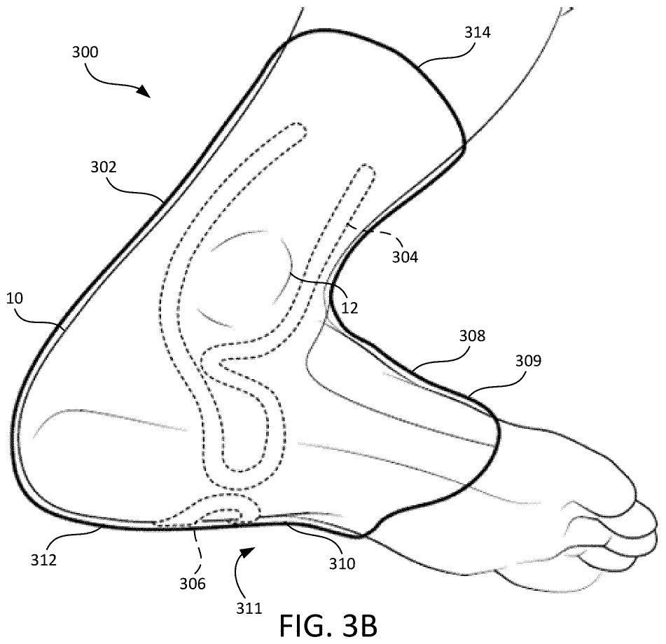

FIGS. 3A and 3B illustrate an ankle brace 300 according to an embodiment of the present disclosure being worn on the foot 10 of a wearer. The ankle brace 300 includes a main body 302 that is configured to receive the foot 10 of the wearer, including the ankle 12, and a first ankle stay 304 and a second ankle stay 306, which may be any of the ankle stays described herein (for example, the ankle stays 120, 122). The main body 302 generally includes the same features, components, shapes, dimensions, and characteristics as the main body 102 of the ankle brace 100, except that the main body 302 includes a first portion 308 and a second portion 310 that have different materials and/or characteristics. The first portion 308 has a first stretchability and the second portion 310 has a second stretchability that is less than the first stretchability. For example, the second stretchability may be 25 percent less than the first stretchability, 50 percent less than the first stretchability, or 75 percent less than the first stretchability. The first portion 308 may be formed of a relatively elastic material, such as neoprene. The second portion 310 may be formed of a relatively inelastic material, such as a woven fabric. The second portion 310 may be present on the first side portion 309 and the second side portion 311 adjacent the ankle stays 304, 306 and other locations of the ankle brace 300 (such as the bottom portion 312 of the main body 302) to facilitate medial/lateral and vertical ankle support. The second portion 310 may be absent and the first portion 308 may be present at other locations of the ankle brace 300 (such as between the bottom portion 312 and the top portion 314 and overlaying the dorsal surface of the foot 10) to provide a compression fit against the foot 10. In some embodiments, the first portion 308 and the second portion 310 partially overlap, for example, adjacent the ankle stays 304, 306 to form pockets (not shown) in which the ankle stays 304, 306 are carried.

As another example of ankle braces according to embodiments of the present disclosure including additional and/or different features, ankle stays of ankle braces according to some embodiments of the present disclosure may have different shapes, dimension, and/or characteristics. As a more specific example, in some embodiments the shapes and/or dimensions of one or more portions of one or both of the ankle stays are different than those described above and shown in the drawings. As another example, in some embodiments the ankle stays could be symmetric over a coronal plane bisecting the ankle of the wearer. Several exemplary embodiments of such ankle stays are described below.

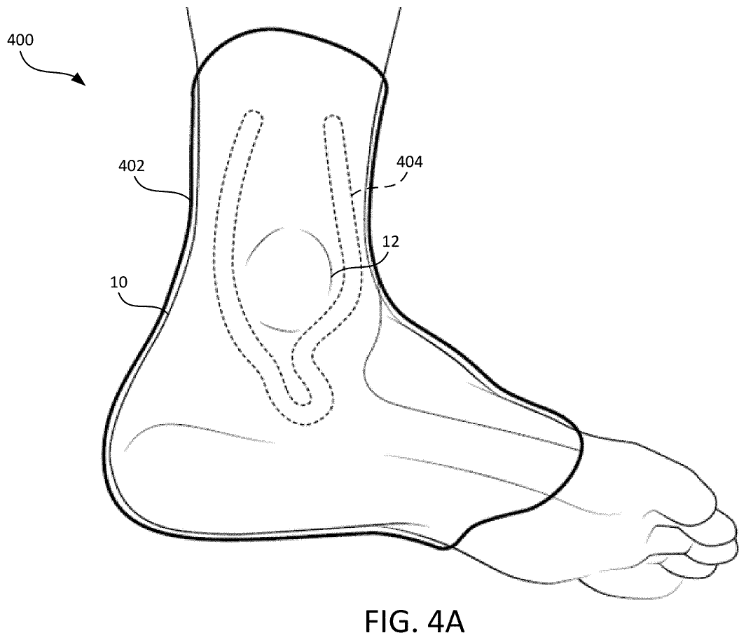

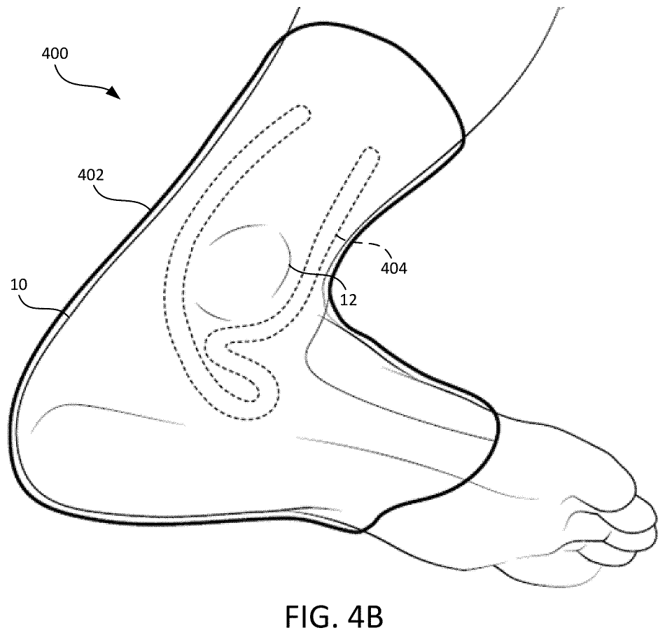

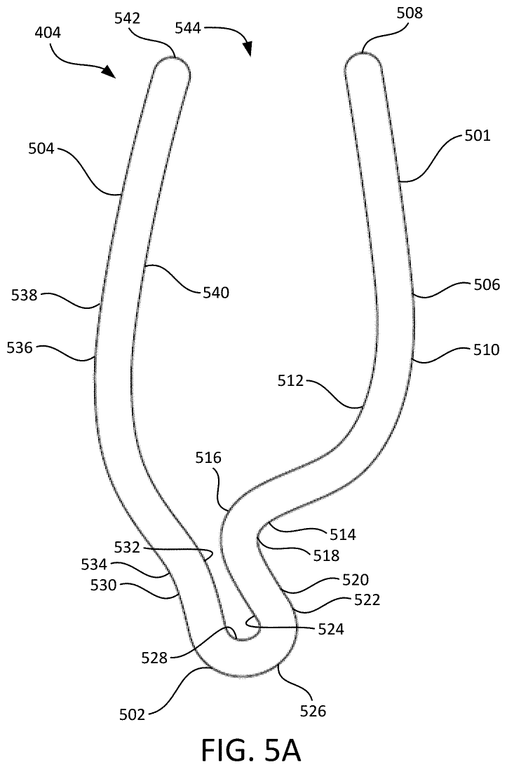

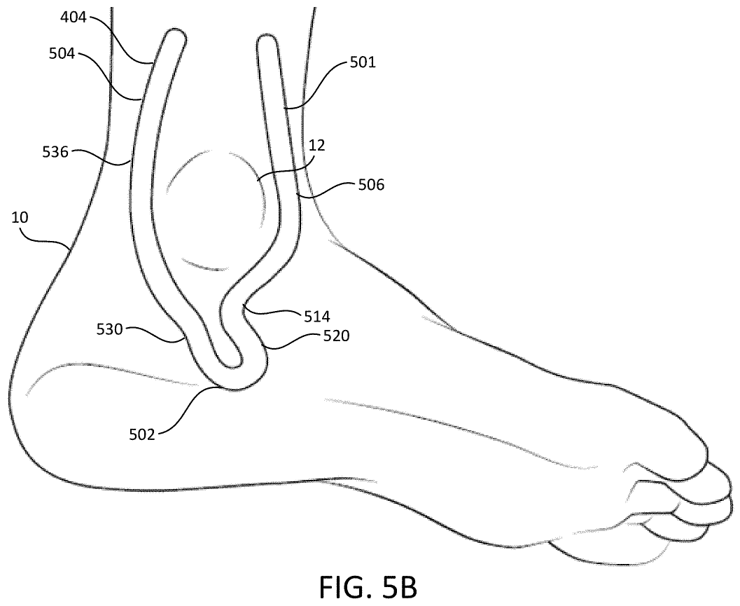

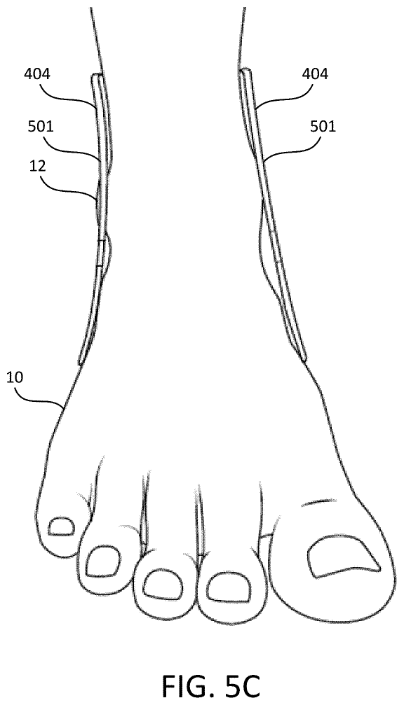

FIGS. 4A and 4B illustrate an ankle brace 400 according to an embodiment of the present disclosure being worn on the foot 10 of a wearer. The ankle brace 400 includes a main body 402 that is configured to receive the foot 10 of the wearer, including the ankle 12. The main body 402 may be, for example, any of the main bodies described herein (for example, the main body 102 or the main body 302, as illustrated). The main body 402 carries ankle stays (a first ankle stay 404 is shown in FIGS. 4A and 4B and a second ankle stay is obscured). FIG. 5A shows the ankle stay 404 in a flat configuration for illustrative purposes. FIG. 5B illustrates the ankle stay 404 positioned relative to the foot 10 of the wearer, and the main body 402 of the ankle brace 400 is hidden for illustrative purposes. FIG. 5C illustrates two of the ankle stays 404 positioned relative to the foot 10 of the wearer, and the main body 402 of the ankle brace 400 is hidden for illustrative purposes. The ankle stay 404 generally includes similar components and features compared to the ankle stays 120, 122, and in the drawings similar components and features are represented by the same reference numbers increased by 400 (that is, the ankle stay 404 includes an anterior portion 501, an inferior portion 502, a posterior portion 504, and so forth). In contrast to the ankle stays 120, 122, however, the inferior anterior section 520, the inferior portion 502, and the inferior posterior section 530 are configured to be disposed to the side of the foot 10 of the wearer (that is, not inferiorly relatively to the sole of the wearer).

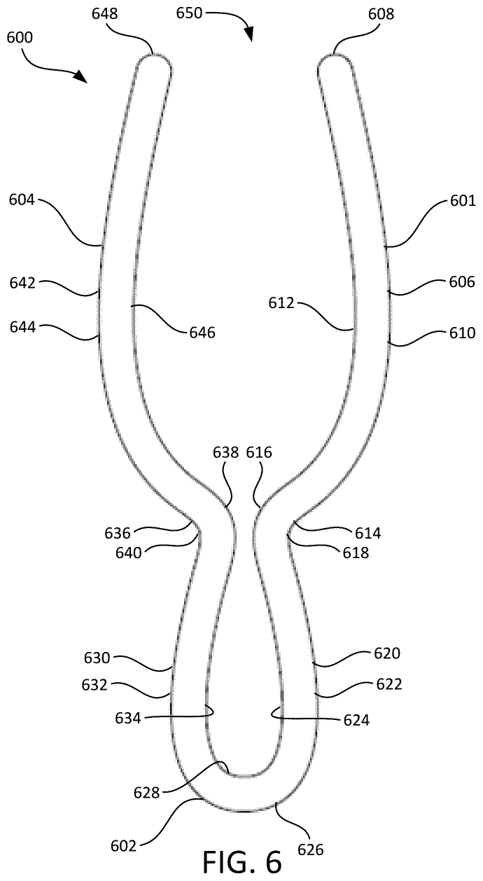

FIG. 6 shows an ankle stay 600 according to an embodiment of the present disclosure, which may be included as part of an ankle brace in lieu of an ankle stay as described above. The ankle stay 600 is shown in a flat configuration for illustrative purposes. The ankle stay 600 is generally similar to the ankle stays 120, 122, although the ankle stay 600 is symmetric over the coronal plane bisecting the ankle of the wearer. That is, the ankle stay 600 generally includes an anterior portion 601, an inferior portion 602, and a posterior portion 604. The anterior portion 601 includes a superior anterior section 606 (configured to be disposed anteriorly relative to the ankle of the wearer and having an anterior end 608, an anteriorly-facing first convex edge 610, and a posteriorly-facing first concave edge 612), an intermediate anterior section 614 (configured to be disposed inferiorly relative to the ankle of the wearer and having a posteriorly-facing second convex edge 616 and an anteriorly-facing second concave edge 618), and an inferior anterior section 620 (configured to be disposed to the side and inferiorly relatively to the sole of the wearer and having an anteriorly-facing third convex edge 622 and a posteriorly-facing third concave edge 624). The inferior portion 602 is configured to be disposed inferiorly relative to the sole of the wearer and includes an outwardly-facing fourth convex edge 626 and an inwardly-facing fourth concave edge 628. The posterior portion 604 includes an inferior posterior section 630 (configured to be disposed to the side and inferiorly relatively to the sole of the wearer and having a posteriorly-facing fifth convex edge 632 and an anteriorly-facing fifth concave edge 634), an intermediate posterior section 636 (configured to be disposed inferiorly relative to the ankle of the wearer and having an anteriorly-facing sixth convex edge 638 and a posteriorly-facing sixth concave edge 640), and a superior posterior section 642 (configured to be disposed posteriorly relative to the ankle of the wearer and having a posteriorly-facing seventh convex edge 644, an anteriorly-facing seventh concave edge 646, and a posterior end 648 disposed posteriorly relative to the anterior end 608 to provide the ankle stay 600 with an open top 650).

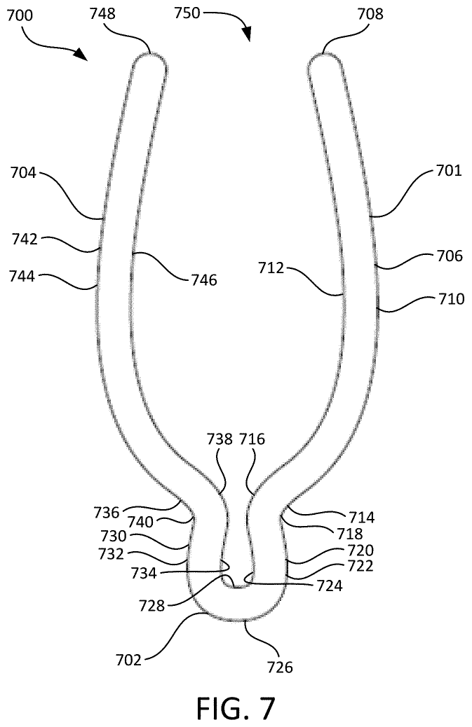

FIG. 7 shows an ankle stay 700 according to an embodiment of the present disclosure, which may be included as part of an ankle brace in lieu of an ankle stay as described above. The ankle stay 700 is shown in a flat configuration for illustrative purposes. The ankle stay 700 generally includes similar components and features compared to the ankle stay 404, and in the drawings similar components and features are represented by the same reference numbers increased by 300 (that is, the ankle stay 700 includes an anterior portion 701, an inferior portion 702, a posterior portion 704, and so forth). In contrast to the ankle stay 404, however, the inferior anterior section 720, the inferior portion 702, and the inferior posterior section 730 are configured to be disposed to the side of the foot of the wearer (that is, not inferiorly relatively to the sole of the wearer).

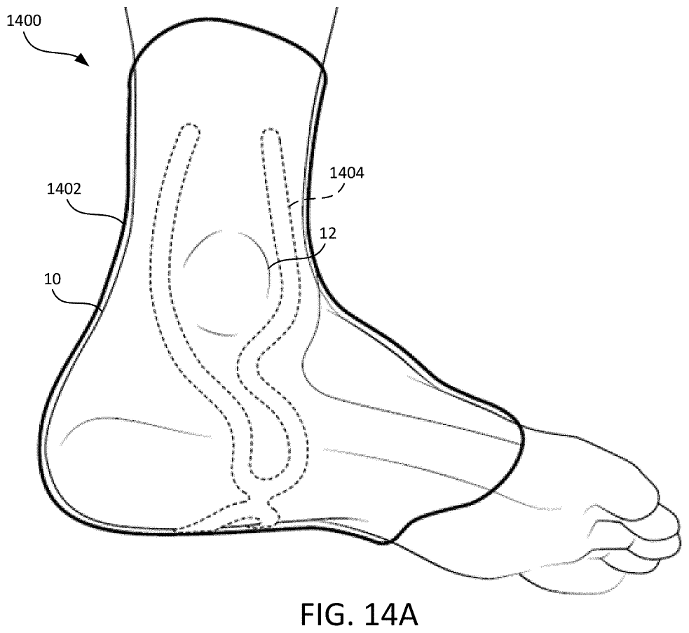

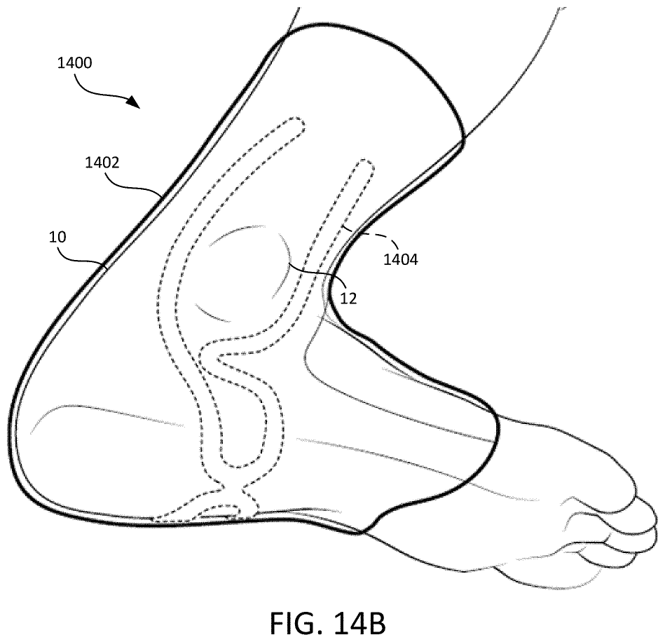

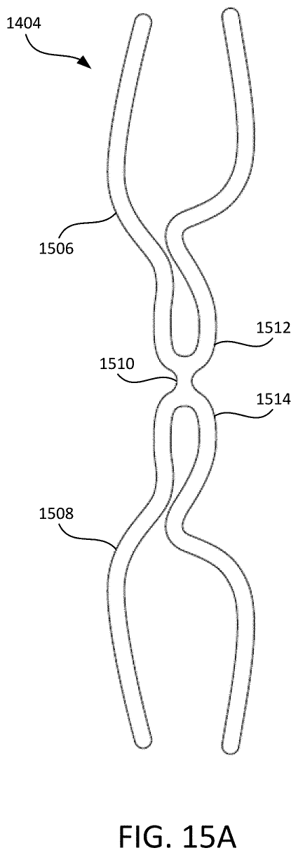

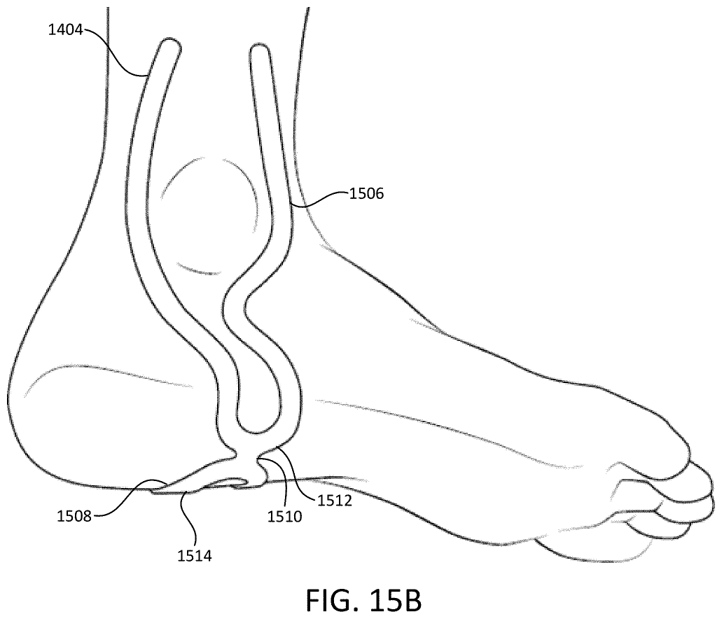

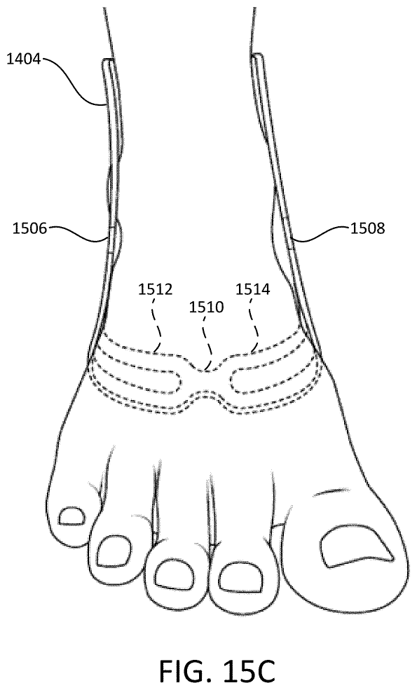

FIGS. 14A and 14B illustrate an ankle brace 1400 according to an embodiment of the present disclosure being worn on the foot 10 of a wearer. The ankle brace 1400 includes a main body 1402 that is configured to receive the foot 10 of the wearer, including the ankle 12. The main body 1402 may be, for example, any of the main bodies described herein (for example, the main body 102 or the main body 302, as illustrated). The main body 1402 carries an ankle stay 1404. FIG. 15A shows the ankle stay 1404 in a flat configuration for illustrative purposes. FIGS. 15B and 15C illustrate the ankle stay 1404 positioned relative to the foot 10 of the wearer, and the main body 1402 of the ankle brace 1400 is hidden for illustrative purposes. The ankle stay 1404 includes a first, or lateral, side portion 1506 and a second, or medial, side portion 1508, both of which may have the same or a similar structure to, for example, any of the ankle stays described herein above (for example and as illustrated, the ankle stays 120, 122). The ankle stay 1404 also includes a connecting portion 1510 that couples an inferior portion 1512 of the first side portion 1506 to an inferior portion 1514 of the second side portion 1508. The connecting portion 1510 is configured to underlie the foot 10 of the wearer. In some embodiments and as illustrated, the first side portion 1506, the second side portion 1508, and the connecting portion 1510 are monolithically formed, although in other embodiments the first side portion 1506, the second side portion 1508, and the connecting portion 1510 may be formed as separate sections that are joined, for example, via adhesives, ultrasonic welding, or the like.

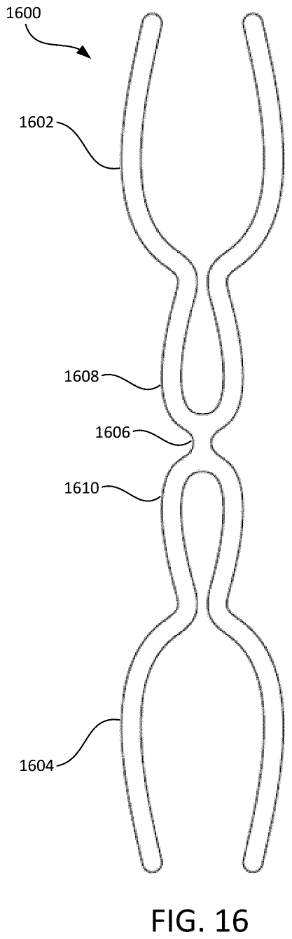

FIG. 16 shows an ankle stay 1600 according to an embodiment of the present disclosure, which may be included as part of an ankle brace in lieu of an ankle stay as described above. The ankle stay 1600 is shown in a flat configuration for illustrative purposes. The ankle stay 1600 is generally similar to the ankle stay 1404, although the ankle stay 1600 is symmetric over the coronal plane bisecting the ankle of the wearer. That is, the ankle stay 1600 includes a first, or lateral, side portion 1602 and a second, or medial, side portion 1604, both of which may have the same or a similar structure to, for example, any of the symmetric ankle stays described herein above (for example and as illustrated, the ankle stay 600). The ankle stay 1600 also includes a connecting portion 1606 that couples an inferior portion 1608 of the first side portion 1602 to an inferior portion 1610 of the second side portion 1604. The connecting portion 1606 is configured to underlie the foot of the wearer. In some embodiments and as illustrated, the first side portion 1602, the second side portion 1604, and the connecting portion 1606 are monolithically formed, although in other embodiments the first side portion 1602, the second side portion 1604, and the connecting portion 1606 may be formed as separate sections that are joined, for example, via adhesives, ultrasonic welding, or the like.

As another example of ankle braces according to embodiments of the present disclosure including additional and/or different features, main bodies of ankle braces according to embodiments of the present disclosure may include additional components and/or features that facilitate supporting the foot of the wearer and/or securement to the foot of the wearer. In some embodiments, for example, main bodies of ankle braces include strap systems (that is, one or more straps) that facilitate supporting the foot of the wearer and/or securement to the foot of the wearer. Several exemplary embodiments of such ankle brace are described below.

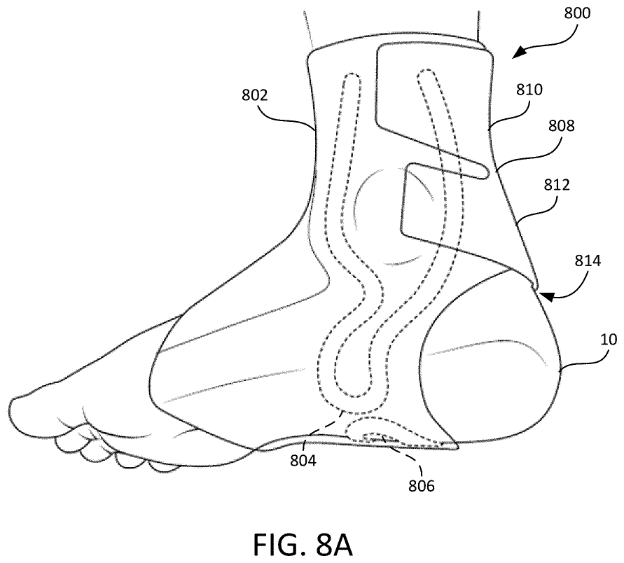

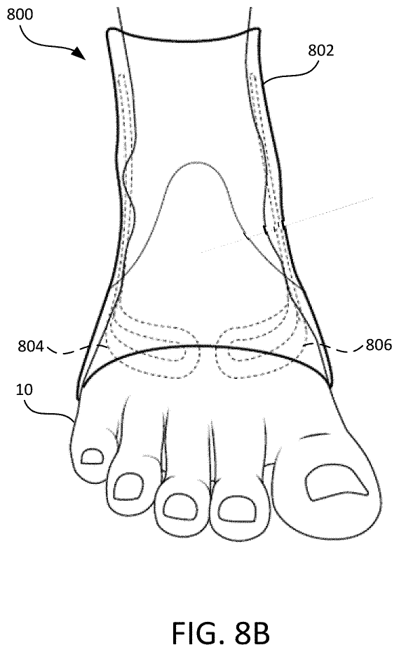

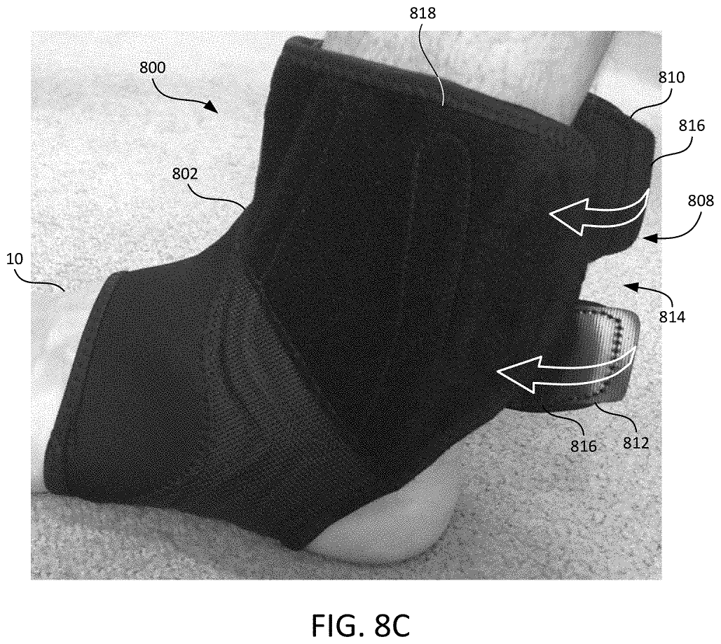

FIGS. 8A-8B illustrate an ankle brace 800 according to an embodiment of the present disclosure being worn on the foot 10 of a wearer. FIG. 8C illustrates the ankle brace 800 being secured to the foot 10 of the wearer. The ankle brace 800 includes a main body 802 that is configured to receive the foot 10 of the wearer. The main body 802 may be, for example, any of the main bodies described herein (for example, the main body 102 or the main body 302, as illustrated). The main body 802 carries a first ankle stay 804 and a second ankle stay 806, which may be, for example, any of the ankle stays described herein (for example, the ankle stays 120, 122 as illustrated, and/or the ankle stays 404, 600, 700). In other embodiments, the main body 802 carries one ankle stay, which may be, for example, the ankle stay 120, 122, 404, 600, 700, 1404, or 1600. The main body 802 carries a strap assembly 808, which includes an upper rear strap 810 and a lower rear strap 812, for selectively closing a rear opening 814 and securing the ankle brace 800 to the foot 10 of the wearer. The upper rear strap 810 and the lower rear strap 812 may be monolithically formed with the main body 802. In some embodiments and as illustrated, the upper rear strap 810 and the lower rear strap 812 detachably couple to another portion of the main body 802 via hook and loop fasteners (for example, the upper rear strap 810 and the lower rear strap 812 carry a plurality of hooks 816 (see FIG. 8C) and the main body 802 carries a plurality of loops 818). In other embodiments, the upper rear strap 810 and the lower rear strap 812 detachably couple to the other portion of the main body 802 in other manners, such as via clasps, buckles, laces, clamps, or the like.

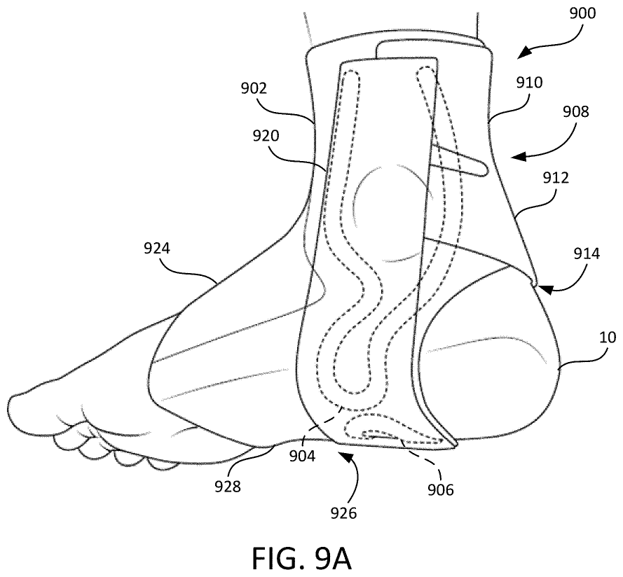

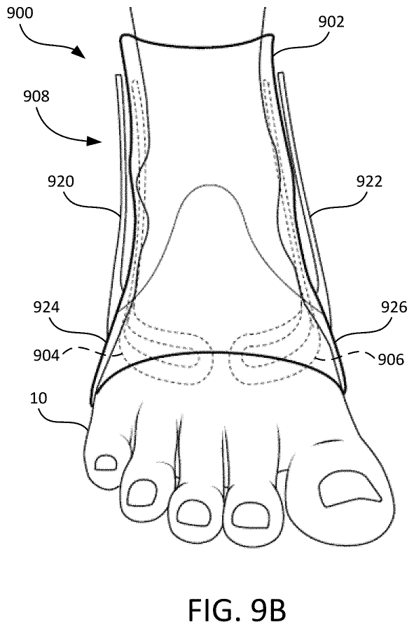

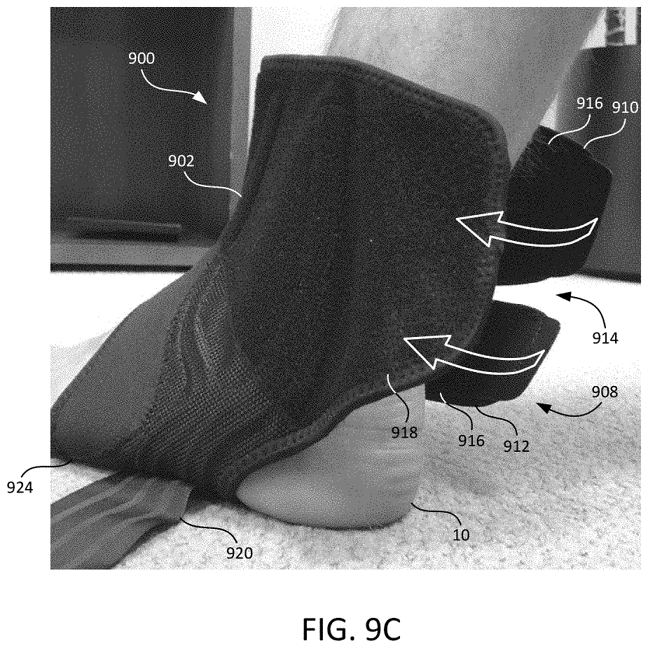

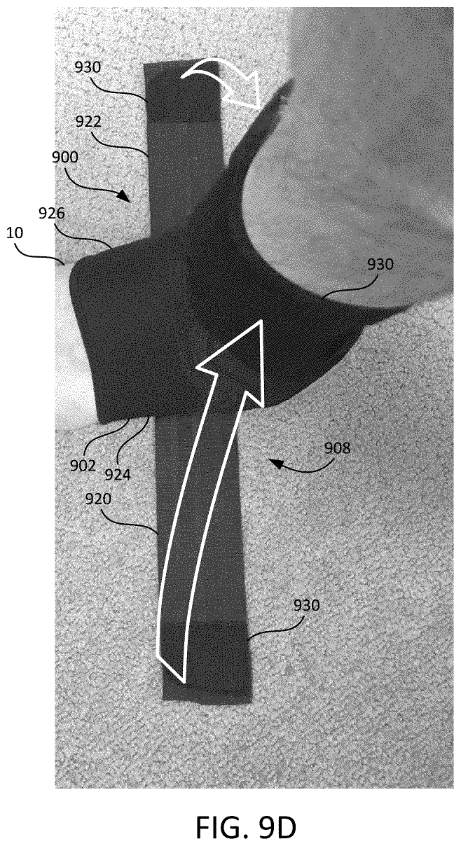

FIGS. 9A-9B illustrate an ankle brace 900 according to an embodiment of the present disclosure being worn on the foot 10 of a wearer. FIGS. 9C-9D illustrate the ankle brace 900 being secured to the foot 10 of the wearer. The ankle brace 900 includes a main body 902 that is configured to receive the foot 10 of the wearer. The main body 902 may be, for example, any of the main bodies described herein (for example, the main body 102 or the main body 302, as illustrated). The main body 902 carries a first ankle stay 904 and a second ankle stay 906, which may be, for example, any of the ankle stays described herein (for example, the ankle stays 120, 122 as illustrated, and/or the ankle stays 404, 600, 700). In other embodiments, the main body 902 carries one ankle stay, which may be, for example, the ankle stay 120, 122, 404, 600, 700, 1404, or 1600.

The main body 902 also carries a strap assembly 908. The strap assembly 908 includes an upper rear strap 910 and a lower rear strap 912 for selectively closing a rear opening 914 and securing the ankle brace 900 to the foot 10 of the wearer. The upper rear strap 910 and the lower rear strap 912 may be monolithically formed with the main body 902. In some embodiments and as illustrated, the upper rear strap 910 and the lower rear strap 912 detachably couple to another portion of the main body 902 via hook and loop fasteners (for example, the upper rear strap 910 and the lower rear strap 912 carry a plurality of hooks 916 (see FIG. 9C) and the main body 902 carries a plurality of loops 918). In other embodiments, the upper rear strap 910 and the lower rear strap 912 detachably couple to the other portion of the main body 902 in other manners, such as via clasps, buckles, laces, clamps, or the like. The strap assembly 908 further includes a first side stirrup strap 920 and a second side stirrup strap 922. The first side stirrup strap 920 and the second side stirrup strap 922 extend from the sole 928 upwardly and along the first side portion 924 and the second side portion 926 of the main body 902, respectively, to support the foot 10 of the wearer and secure the ankle brace 900 to the foot 10 of the wearer. In some embodiments, the first side stirrup strap 920 and the second side stirrup strap 922 are monolithically formed with each other. In some embodiments, the first side stirrup strap 920 and the second side stirrup strap 922 couple to the sole 928 of the main body 902 via stitching (not shown), adhesives (not shown), or the like. In some embodiments and as illustrated, the first side stirrup strap 920 and the second side stirrup strap 922 detachably couple to another portion of the main body 902 via hook and loop fasteners (for example, the first side stirrup strap 920 and the second side stirrup strap 922 carry a plurality of hooks 930 (see FIG. 9D) that detachably couple to the plurality of loops 918). In other embodiments, the first side stirrup strap 920 and the second side stirrup strap 922 detachably couple to the other portion of the main body 902 in other manners, such as via clasps, buckles, laces, clamps, or the like.

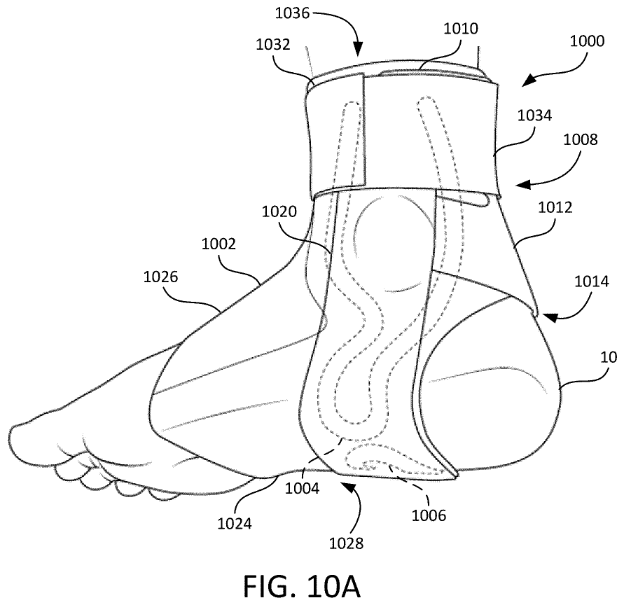

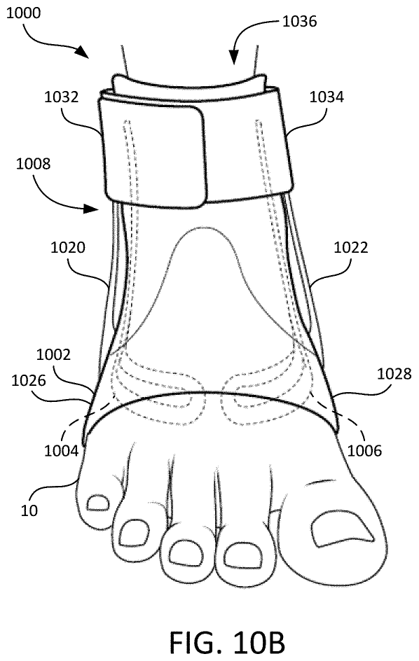

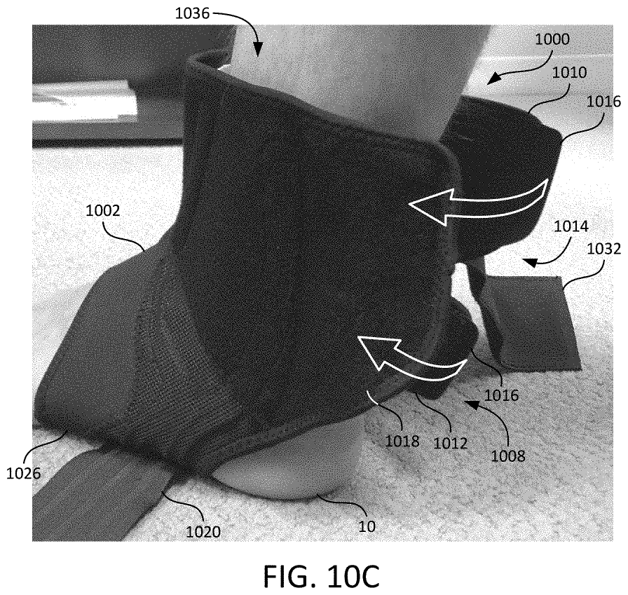

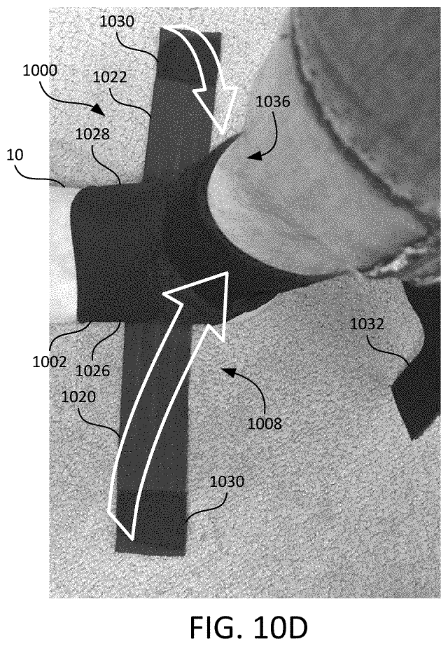

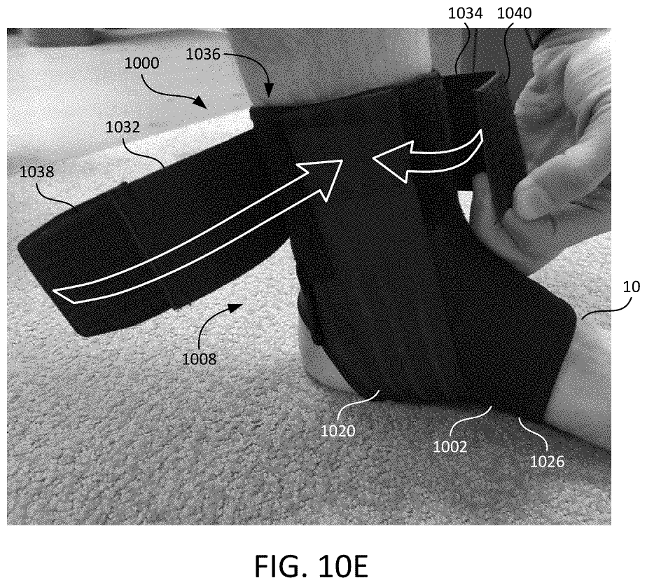

FIGS. 10A-10B illustrate an ankle brace 1000 according to an embodiment of the present disclosure being worn on the foot 10 of a wearer. FIGS. 10C-10E illustrate the ankle brace 1000 being secured to the foot 10 of the wearer. The ankle brace 1000 includes a main body 1002 that is configured to receive the foot 10 of the wearer. The main body 1002 may be, for example, any of the main bodies described herein (for example, the main body 102 or the main body 302, as illustrated). The main body 1002 carries a first ankle stay 1004 and a second ankle stay 1006, which may be, for example, any of the ankle stays described herein (for example, the ankle stays 120, 122 as illustrated, and/or the ankle stays 404, 600, 700). In other embodiments, the main body 1002 carries one ankle stay, which may be, for example, the ankle stay 120, 122, 404, 600, 700, 1404, or 1600).

The main body 1002 also carries a strap assembly 1008. The strap assembly 1008 includes an upper rear strap 1010 and a lower rear strap 1012 for selectively closing a rear opening 1014 and securing the ankle brace 1000 to the foot 10 of the wearer. The upper rear strap 1010 and the lower rear strap 1012 may be monolithically formed with the main body 1002. In some embodiments and as illustrated, the upper rear strap 1010 and the lower rear strap 1012 detachably couple to another portion of the main body 1002 via hook and loop fasteners (for example, the upper rear strap 1010 and the lower rear strap 1012 carry a plurality of hooks 1016 (see FIG. 10C) and the main body 1002 carries plurality of loops 1018). In other embodiments, the upper rear strap 1010 and the lower rear strap 1012 detachably couple to the other portion of the main body 1002 in other manners, such as via clasps, buckles, laces, clamps, or the like. The strap assembly 1008 further includes a first side stirrup strap 1020 and a second side stirrup strap 1022. The first side stirrup strap 1020 and the second side stirrup strap 1022 extend from the sole 1024 upwardly and along the first side portion 1026 and the second side portion 1028 of the main body 1002, respectively, to support the foot 10 of the wearer and secure the ankle brace 1000 to the foot 10 of the wearer. In some embodiments, the first side stirrup strap 1020 and the second side stirrup strap 1022 are monolithically formed with each other. In some embodiments, the first side stirrup strap 1020 and the second side stirrup strap 1022 couple to the sole 1024 of the main body 1002 via stitching (not shown), adhesives (not shown), or the like. In some embodiments and as illustrated, the first side stirrup strap 1020 and the second side stirrup strap 1022 detachably couple to another portion of the main body 1002 via hook and loop fasteners (for example, the first side stirrup strap 1020 and the second side stirrup strap 1022 carry a plurality of hooks 1030 (see FIG. 10D) that detachably couple to the plurality of loops 1018). In other embodiments, the first side stirrup strap 1020 and the second side stirrup strap 1022 detachably couple to the other portion of the main body 1002 in other manners, such as via clasps, buckles, laces, clamps, or the like. The strap assembly 1008 further includes a first upper wrapping strap 1032 and a second upper wrapping strap 1034 to secure the ankle brace 1000 to the foot 10 of the wearer. The first upper wrapping strap 1032 and the second upper wrapping strap 1034 extend from the upper rear strap 1010, around the main body 1002 adjacent the top opening 1036, and the first upper wrapping strap 1032 overlies and is detachably coupled to the second upper wrapping strap 1034. In some embodiments, the first upper wrapping strap 1032 and the second upper wrapping strap 1034 are monolithically formed with each other. In some embodiments, the first upper wrapping strap 1032 and the second upper wrapping strap 1034 couple to the upper rear strap 1010 via stitching (not shown), adhesives (not shown), or the like. In some embodiments and as illustrated, the first upper wrapping strap 1032 and the second upper wrapping strap 1034 detachably couple to each other via hook and loop fasteners (for example and as shown in FIG. 10E, the first upper wrapping strap 1032 carries a plurality of hooks 1038 and the second upper wrapping strap 1034 carries a plurality of loops 1040). In other embodiments, the first upper wrapping strap 1032 and the second upper wrapping strap 1034 detachably couple to each other in other manners, such as via clasps, buckles, laces, clamps, or the like.

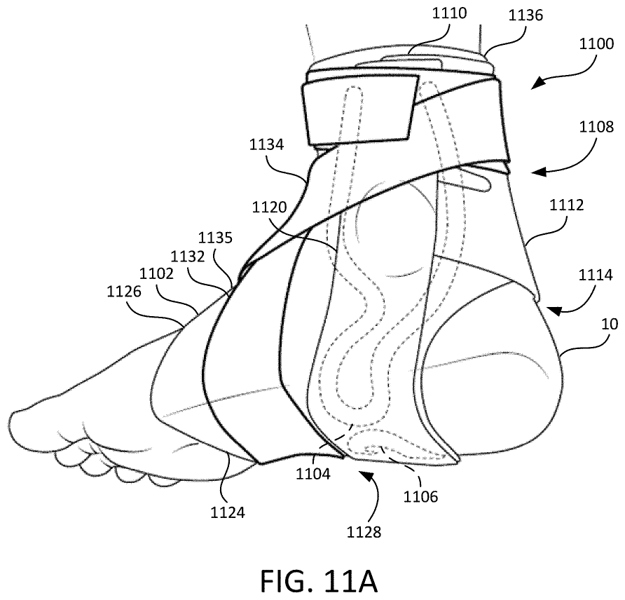

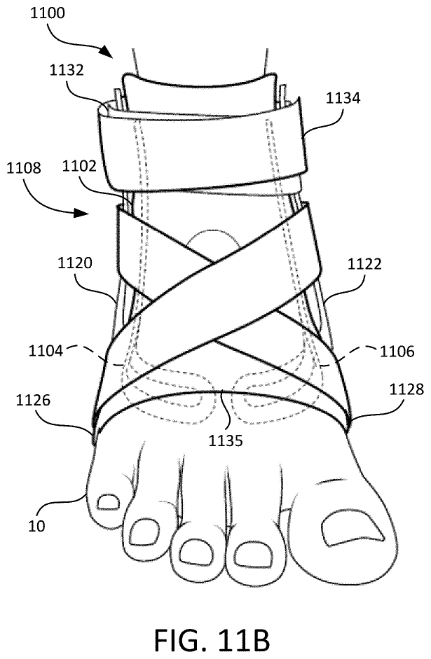

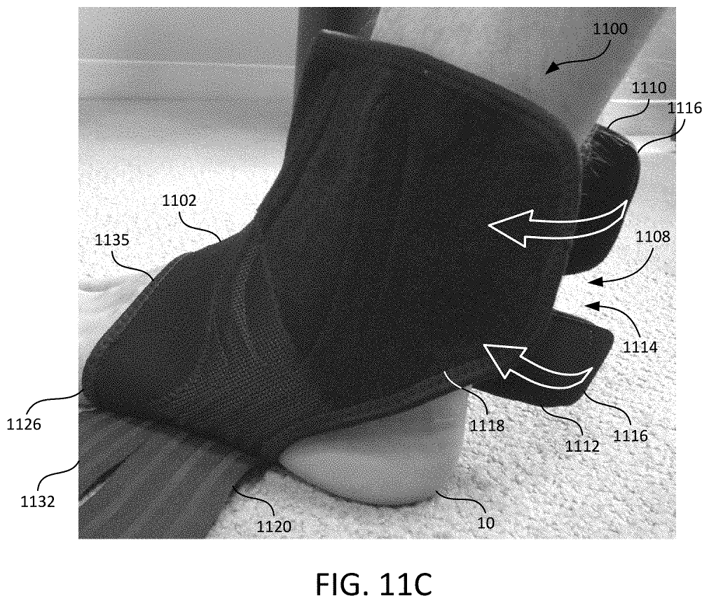

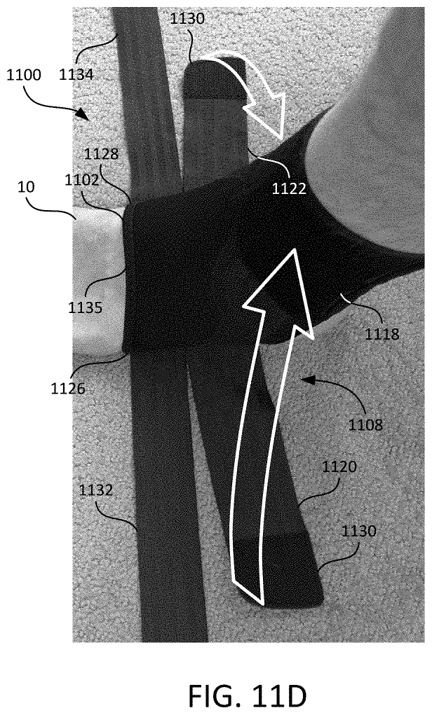

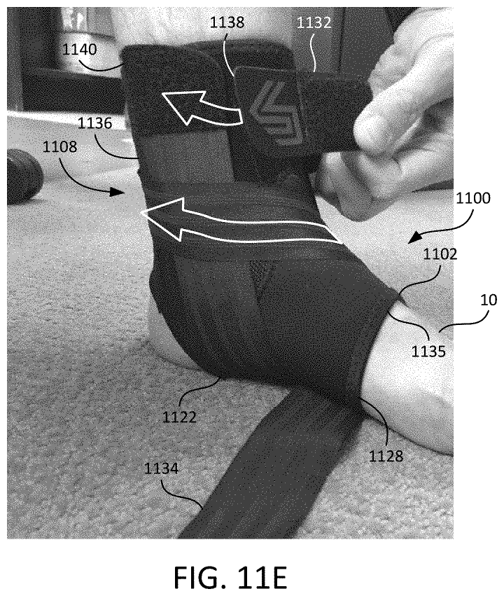

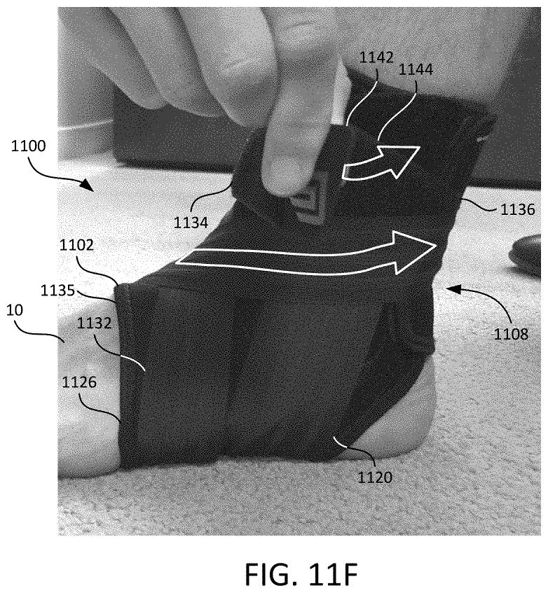

FIGS. 11A-11B illustrate an ankle brace 1100 according to an embodiment of the present disclosure being worn on the foot 10 of a wearer. FIGS. 11C-11F illustrate the ankle brace 1100 being secured to the foot 10 of the wearer. The ankle brace 1100 includes a main body 1102 that is configured to receive the foot 10 of the wearer. The main body 1102 may be, for example, any of the main bodies described herein (for example, the main body 102, as illustrated, or the main body 302). The main body 1102 carries a first ankle stay 1104 and a second ankle stay 1106, which may be, for example, any of the ankle stays described herein (for example, the ankle stays 120, 122 as illustrated, and/or the ankle stays 404, 600, 700). In other embodiments, the main body 1102 carries one ankle stay, which may be, for example, the ankle stay 120, 122, 404, 600, 700, 1404, or 1600).

The main body 1102 also carries a strap assembly 1108. The strap assembly 1108 includes an upper rear strap 1110 and a lower rear strap 1112 for selectively closing a rear opening 1114 and securing the ankle brace 1100 to the foot 10 of the wearer. The upper rear strap 1110 and the lower rear strap 1112 may be monolithically with the main body 1102. In some embodiments and as illustrated, the upper rear strap 1110 and the lower rear strap 1112 detachably couple to another portion of the main body 1102 via hook and loop fasteners (for example, the upper rear strap 1110 and the lower rear strap 1112 carry a plurality of hooks 1116 (see FIG. 11C) and the main body 1102 carries a plurality of loops 1118). In other embodiments, the upper rear strap 1110 and the lower rear strap 1112 detachably couple to the other portion of the main body 1102 in other manners, such as via clasps, buckles, laces, clamps, or the like. The strap assembly 1108 further includes a first side stirrup strap 1120 and a second side stirrup strap 1122. The first side stirrup strap 1120 and the second side stirrup strap 1122 extend from the sole 1124 upwardly and along the first side portion 1126 and the second side portion 1128 of the main body 1102, respectively, to support the foot 10 of the wearer and secure the ankle brace 1100 to the foot 10 of the wearer. In some embodiments, the first side stirrup strap 1120 and the second side stirrup strap 1122 are monolithically formed with each other. In some embodiments, the first side stirrup strap 1120 and the second side stirrup strap 1122 couple to the sole 1124 of the main body 1102 via stitching (not shown), adhesives (not shown), or the like. In some embodiments and as illustrated, the first side stirrup strap 1120 and the second side stirrup strap 1122 detachably couple to another portion of the main body 1102 via hook and loop fasteners (for example, the first side stirrup strap 1120 and the second side stirrup strap 1122 carry a plurality of hooks 1130 (see FIG. 11D) that detachably couple to plurality of loops 1118). In other embodiments, the first side stirrup strap 1120 and the second side stirrup strap 1122 detachably couple to the other portion of the main body 1102 in other manners, such as via clasps, buckles, laces, clamps, or the like. The strap assembly 1108 further includes a first crossing strap 1132 and a second crossing strap 1134 to support the foot 10 of the wearer and secure the ankle brace 1100 to the foot 10 of the wearer. The first crossing strap 1132 and the second crossing strap 1134 extend in opposite directions from the sole 1124, extend superiorly and cross each other at the portion of the top portion 1135 of the main body 1102 overlaying the dorsal surface of the foot 10, extend posteriorly and cross each other again at the rear portion 1136 of the main body 1102, and extend anteriorly such that the second crossing strap 1134 then overlies the first crossing strap 1132. In some embodiments, the first crossing strap 1132 and the second crossing strap 1134 are monolithically formed with each other. In some embodiments, the first crossing strap 1132 and the second crossing strap 1134 couple to the sole 1124 via stitching (not shown), adhesives (not shown), or the like. In some embodiments and as illustrated in FIGS. 11A and 11B, the first crossing strap 1132 and the second crossing strap 1134 have a sufficient length to terminate directly above the ankle on opposite sides of the foot 10. More specifically, the first crossing strap 1132 may terminate directly above the ankle on one of the medial side and the lateral side of the foot 10, and the second crossing strap 1134 may terminate directly above the ankle on the other of the medial side and the lateral side of the foot 10. In some embodiments, one of the first crossing strap 1132 and the second crossing strap 1134 detachably couples to the main body 1102 (for example, via hooks and loops), and the other of the first crossing strap 1132 and the second crossing strap 1134 partially overlies and detachably couples to the first of the first crossing strap 1132 and the second crossing strap 1134 (for example, via hooks and loops). In these embodiments, the first side stirrup strap 1120 and the second side stirrup strap 1122 may be shorter than illustrated to permit the first crossing strap 1132 or the second crossing strap 1134 to detachably couple to the main body 1102. In some embodiments, by extending around the leg of the wearer above the ankle, the first crossing strap 1132 and the second crossing strap 1134 provide the same type of support as one or more upper wrapping straps (for example, the first upper wrapping strap 1032 and a second upper wrapping strap 1034; see FIGS. 10A-10E). In some embodiments and as illustrated in FIG. 11E, the first crossing strap 1132 detachably couples to second side stirrup strap 1122 (for example and as shown in FIG. 11E, the first crossing strap 1132 carries a plurality of hooks 1138 and the second side stirrup strap 1122 carries a plurality of loops 1140). In some embodiments and as illustrated in FIG. 11F, the second crossing strap 1134 detachably couples to the first side stirrup strap 1120 (for example and as shown in FIG. 11F, the second crossing strap 1134 carries a plurality of hooks 1142 and the first side stirrup strap 1120 carries a plurality of loops 1144). In other embodiments, the first crossing strap 1132 and the second crossing strap 1134 detachably couple to the second side stirrup strap 1122 and the first side stirrup strap 1120, respectively, in other manners, such as via clasps, buckles, laces, clamps, or the like. In other embodiments, one or more of the straps, such as the first crossing strap 1132 and the second crossing strap 1134, have different lengths than those illustrated.

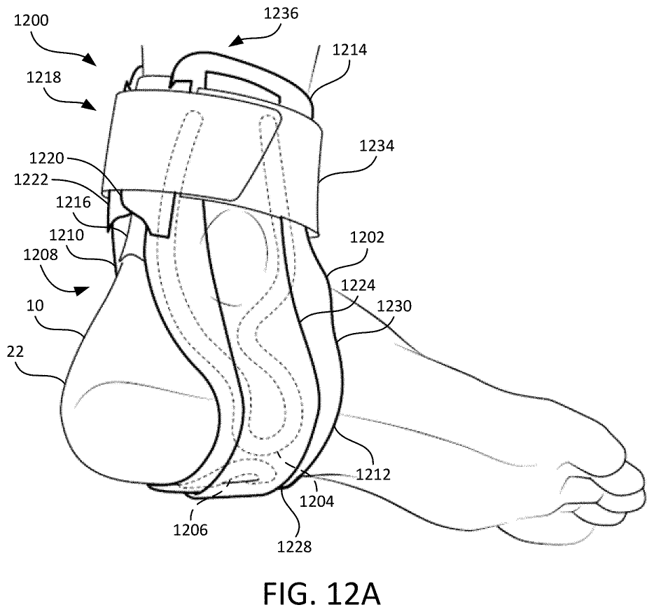

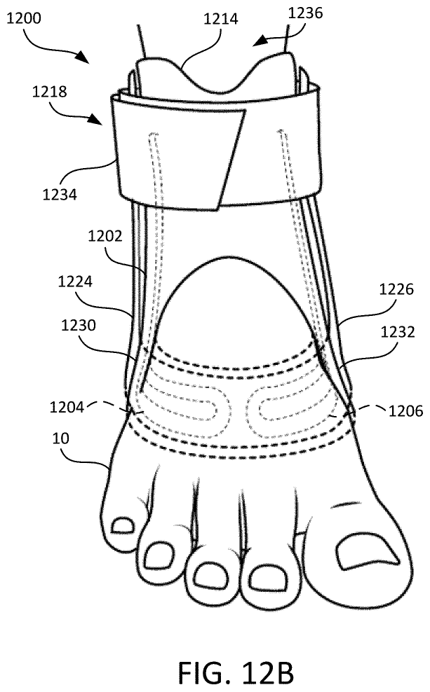

FIGS. 12A-12B illustrate an ankle brace 1200 according to an embodiment of the present disclosure being worn on the foot 10 of a wearer. The ankle brace 1200 includes a main body 1202 that is configured to receive the foot 10 of the wearer. The main body 1202 carries a first ankle stay 1204 and a second ankle stay 1206, which may be, for example, any of the ankle stays described herein (for example, the ankle stays 120, 122 as illustrated, and/or the ankle stays 404, 600, 700). In other embodiments, the main body 1202 carries one ankle stay, which may be, for example, the ankle stay 120, 122, 404, 600, 700, 1404, or 1600). The main body 1202 defines a rear opening 1208 at the rear portion 1210 that extends from the bottom portion 1212 to the top portion 1214. The heel 22 of the foot 10 extends through the rear opening 1208 when the ankle brace 1200 is worn. The rear opening 1208 is also spanned by a bridge 1216 adjacent to the top portion 1214. The bridge 1216 may be formed of a relatively elastic material, such as neoprene, although materials may be additionally or alternatively used.

The main body 1202 also carries a strap assembly 1218. The strap assembly 1218 includes a first rear strap 1220 and a second rear strap 1222 for securing the ankle brace 1200 to the foot 10 of the wearer. In some embodiments, the first rear strap 1220 and the second rear strap 1222 couple to the main body 1202 via stitching (not shown), adhesives (not shown), or the like. In some embodiments, the first rear strap 1220 and the second rear strap 1222 detachably couple to each other via hook and loop fasteners (not shown—for example, the first rear strap 1220 carries a plurality of hooks and the second rear strap 1222 carries a plurality of loops). In other embodiments, the first rear strap 1220 and the second rear strap 1222 detachably couple to each other in other manners, such as via clasps, buckles, laces, clamps, or the like. The strap assembly 1218 further includes a first side stirrup strap 1224 and a second side stirrup strap 1226. The first side stirrup strap 1224 and the second side stirrup strap 1226 extend from the sole 1228 upwardly and along the first side portion 1230 and the second side portion 1232 of the main body 1202, respectively, to support the foot 10 of the wearer and secure the ankle brace 1200 to the foot 10 of the wearer. In some embodiments, the first side stirrup strap 1224 and the second side stirrup strap 1226 are monolithically formed with each other. In some embodiments, the first side stirrup strap 1224 and the second side stirrup strap 1226 couple to the sole 1228 of the main body 1202 via stitching (not shown), adhesives (not shown), or the like. In some embodiments and as illustrated, the first side stirrup strap 1224 and the second side stirrup strap 1226 detachably couple to another portion of the main body 1202 via hook and loop fasteners (for example, the first side stirrup strap 1224 and the second side stirrup strap 1226 carry a plurality of hooks (not shown) that detachably couple to a plurality of loops (not shown) carried on the main body 1202). In other embodiments, the first side stirrup strap 1224 and the second side stirrup strap 1226 detachably couple to the other portion of the main body 1202 in other manners, such as via clasps, buckles, laces, clamps, or the like. The strap assembly 1218 further includes an upper wrapping strap 1234 to secure the ankle brace 1200 to the foot 10 of the wearer. The upper wrapping strap 1234 extends around the main body 1202 adjacent the top opening 1236 and is detachably coupled to itself. In some embodiments, the upper wrapping strap 1234 couples to the main body 1202 via stitching (not shown), adhesives (not shown), or the like. In some embodiments and as illustrated, the upper wrapping strap 1234 detachably couples to itself via hook and loop fasteners (for example, a first portion of the upper wrapping strap 1234 carries a plurality of hooks (not shown) and a second portion of the upper wrapping strap 1234 carries a plurality of loops (not shown)). In other embodiments, the upper wrapping strap 1234 detachably couples to itself in other manners, such as via clasps, buckles, laces, clamps, or the like. In some embodiments, the upper wrapping strap 1234 may be replaced with two upper wrapping straps (for example, the first upper wrapping strap 1032 and a second upper wrapping strap 1034; see FIGS. 10A-10E).

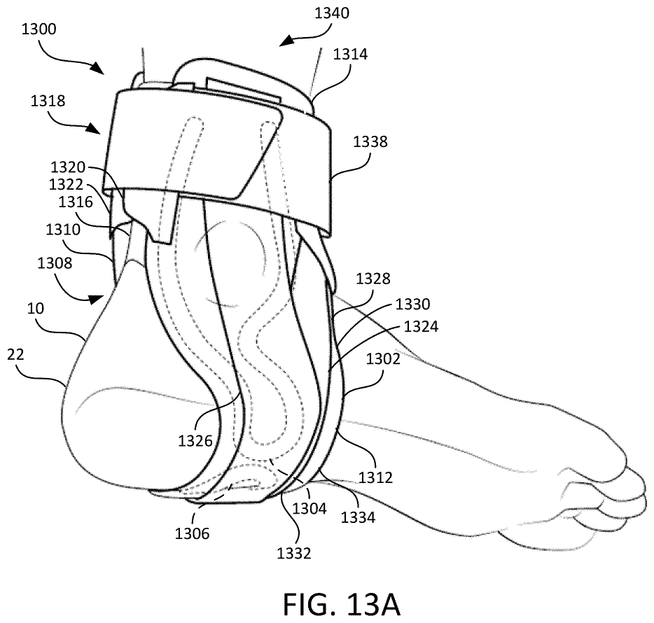

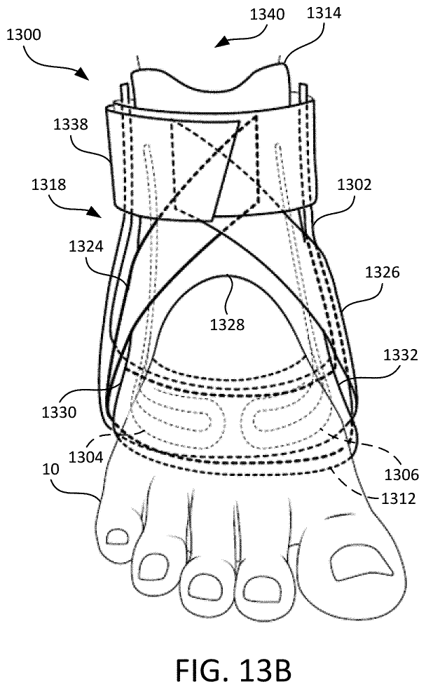

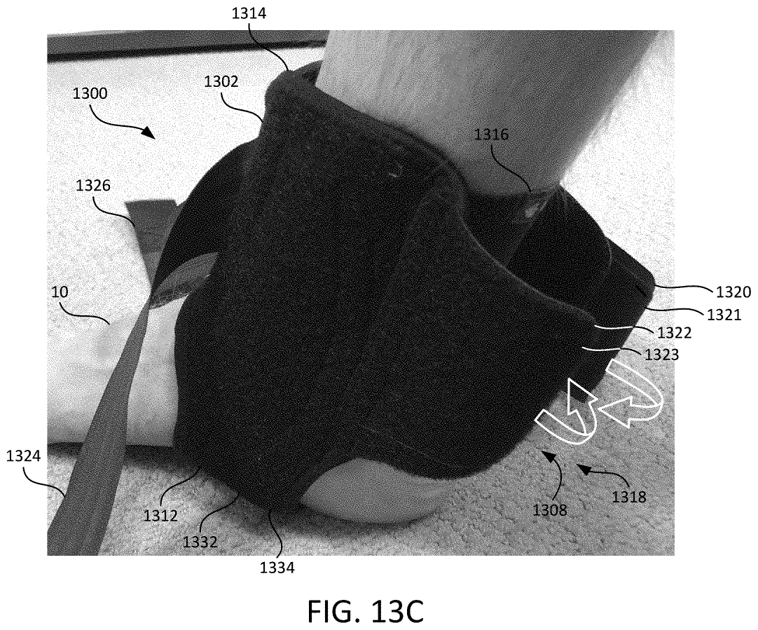

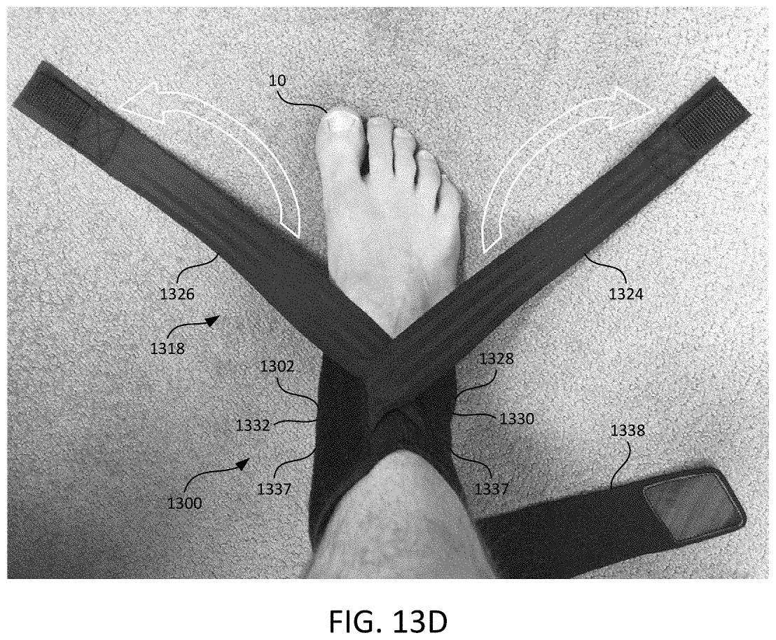

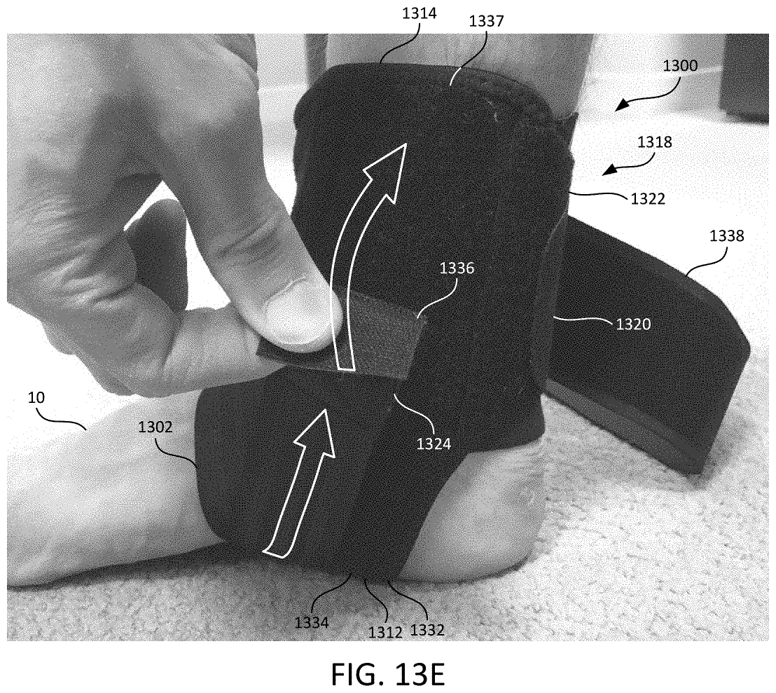

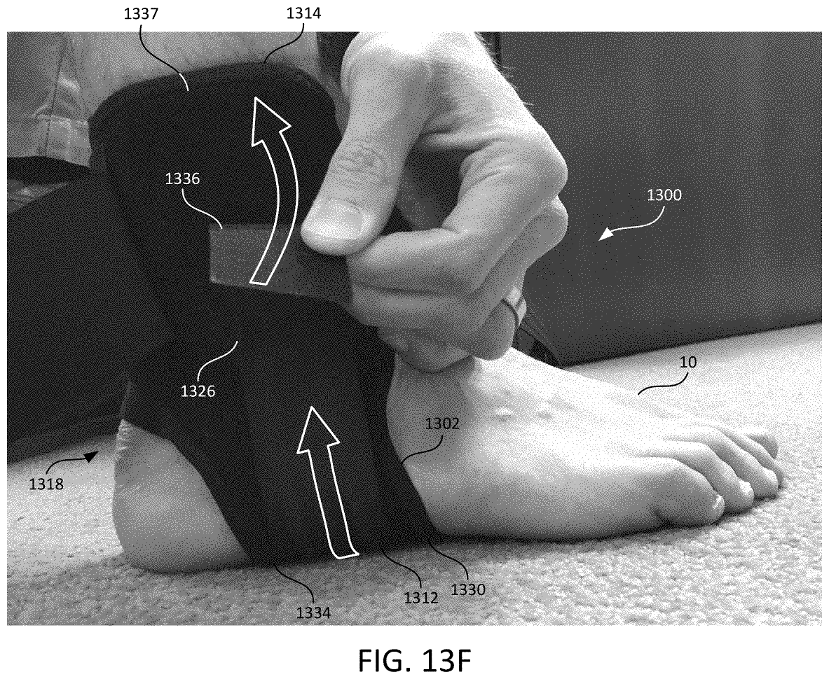

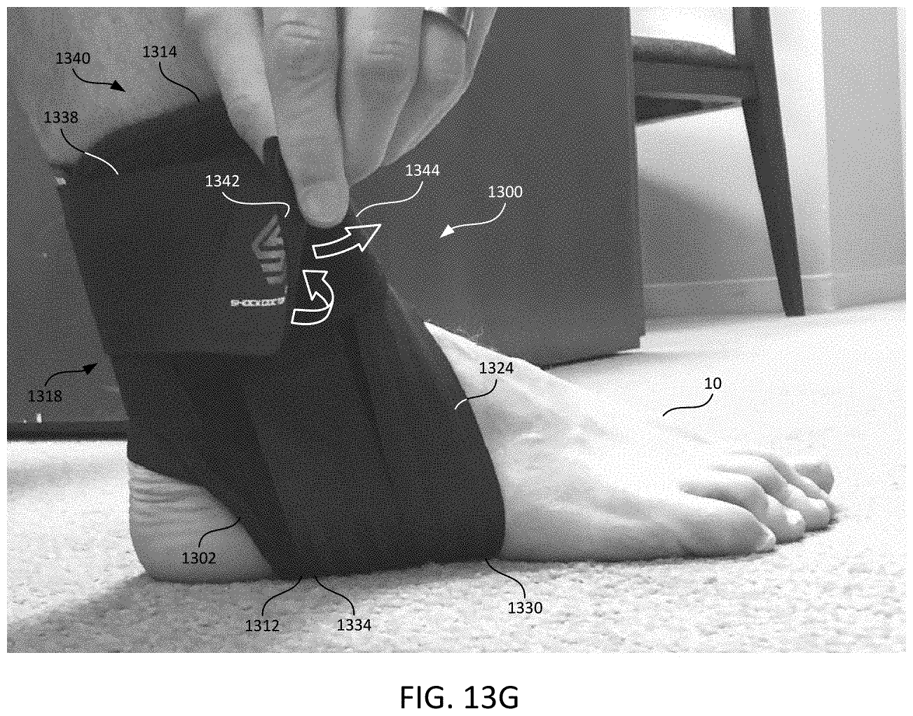

FIGS. 13A-13B illustrate an ankle brace 1300 according to an embodiment of the present disclosure being worn on the foot 10 of a wearer. FIGS. 13C-13G illustrate the ankle brace 1300 being secured to the foot 10 of the wearer. The ankle brace 1300 includes a main body 1302 that is configured to receive the foot 10 of the wearer. The main body 1302 carries a first ankle stay 1304 and a second ankle stay 1306, which may be, for example, any of the ankle stays described herein (for example, the ankle stays 130, 132 as illustrated, and/or the ankle stays 404, 600, 700). In other embodiments, the main body 1302 carries one ankle stay, which may be, for example, the ankle stay 120, 122, 404, 600, 700, 1404, or 1600). The main body 1302 defines a rear opening 1308 at the rear portion 1310 that extends from the bottom portion 1312 to the top portion 1314. The heel 22 of the foot 10 extends through the rear opening 1308 when the ankle brace 1300 is worn. The rear opening 1308 is also spanned by a bridge 1316 adjacent to the top portion 1314. The bridge 1316 may be formed of a relatively elastic material, such as neoprene, although materials may be additionally or alternatively used.

The main body 1302 also carries a strap assembly 1318. The strap assembly 1318 includes a first rear strap 1320 and a second rear strap 1322 for securing the ankle brace 1300 to the foot 10 of the wearer. In some embodiments, the first rear strap 1320 and the second rear strap 1322 couple to the main body 1302 via stitching (not shown), adhesives (not shown), or the like. In some embodiments, the first rear strap 1320 and the second rear strap 1322 detachably couple to each other via hook and loop fasteners (for example, the first rear strap 1320 carries a plurality of hooks 1321 (FIG. 13C) and the second rear strap 1322 carries a plurality of loops 1323). In other embodiments, the first rear strap 1320 and the second rear strap 1322 detachably couple to each other in other manners, such as via clasps, buckles, laces, clamps, or the like. The strap assembly 1318 further includes a first crossing stirrup strap 1324 and a second crossing stirrup strap 1326. The first crossing stirrup strap 1324 and the second crossing stirrup strap 1326 extend from and cross each other on the front portion 1328 of the main body 1302, extend inferiorly along the second side portion 1332 and the first side portion 1330 of the main body 1302, respectively, extend under the sole 1334 and cross each other again, and then extend superiorly along the first side portion 1330 and the second side portion 1332 of the main body 1302, respectively, to support the foot 10 of the wearer and secure the ankle brace 1300 to the foot 10 of the wearer. In some embodiments, the first crossing stirrup strap 1324 and the second crossing stirrup strap 1326 couple to the front portion 1328 of the main body 1302 via stitching (not shown), adhesives (not shown), or the like. In some embodiments and as illustrated, the first crossing stirrup strap 1324 and the second crossing stirrup strap 1326 detachably couple to another portion of the main body 1302 via hook and loop fasteners (for example, the first crossing stirrup strap 1324 and the second crossing stirrup strap 1326 carry a plurality of hooks 1336 (see FIGS. 13E and 13F) that detachably couple to a plurality of loops 1337 carried on the main body 1302). In other embodiments, the first crossing stirrup strap 1324 and the second crossing stirrup strap 1326 detachably couple to the other portion of the main body 1302 in other manners, such as via clasps, buckles, laces, clamps, or the like. In some embodiments, applying sufficient tension to the first crossing stirrup strap 1324 and the second crossing stirrup strap 1326 causes the first ankle stay 1304 and the second ankle stay 1306 to engage each other under the foot 10 of the wearer, which provides additional support to the foot 10 of the wearer. The strap assembly 1318 further includes an upper wrapping strap 1338 to secure the ankle brace 1300 to the foot 10 of the wearer. The upper wrapping strap 1338 extends around the main body 1302 adjacent the top opening 1340 and is detachably coupled to itself. In some embodiments, the upper wrapping strap 1338 couples to the main body 1302 via stitching (not shown), adhesives (not shown), or the like. In some embodiments and as illustrated, the upper wrapping strap 1338 detachably couples to itself via hook and loop fasteners (for example, a first portion of the upper wrapping strap 1338 carries a plurality of hooks 1342 (see FIG. 13G) and a second portion of the upper wrapping strap 1338 carries a plurality of loops 1344). In other embodiments, the upper wrapping strap 1338 detachably couples to itself in other manners, such as via clasps, buckles, laces, clamps, or the like. In some embodiments, the upper wrapping strap 1338 may be replaced with two upper wrapping straps (for example, the first upper wrapping strap 1032 and a second upper wrapping strap 1034; see FIGS. 10A-10E).

Embodiments of ankle braces according to the present disclosure may vary from those described above and shown in the drawings in other manners. For example, embodiments of ankle braces according to the present disclosure may include various combinations of features described herein or lack one or more features described herein. As a specific example, embodiments of ankle braces according to the present disclosure may lack any ankle stays.

Various other modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

The following is claimed:

1. An ankle brace comprising:
a main body configured to receive a foot of a wearer;
an ankle stay coupled to the main body, the ankle stay comprising:
a superior anterior section configured to be disposed anteriorly relative to the ankle of the foot of the wearer, the superior anterior section comprising a first convex edge facing in an anterior direction and a first concave edge facing in a posterior direction;
an intermediate anterior section coupled to the superior anterior section, the intermediate anterior section configured to be disposed inferiorly relative to the ankle of the foot of the wearer, the intermediate anterior section comprising a second convex edge facing in the posterior direction and a second concave edge facing in the anterior direction; and
an inferior anterior section coupled to the intermediate anterior section opposite the superior anterior section.

2. The ankle brace of claim 1, wherein the ankle stay further comprises a superior posterior section configured to be disposed posteriorly relative to the ankle of the foot of the wearer, the superior posterior section comprising a third convex edge facing in the posterior direction and a third concave edge facing in the anterior direction.

3. The ankle brace of claim 2, wherein the ankle stay further comprises an inferior posterior section, the inferior posterior section comprising a fourth convex edge facing in the anterior direction and a fourth concave edge facing in the posterior direction.

4. The ankle brace of claim 3, wherein the ankle stay further comprises an inferior portion, the inferior portion coupling the inferior anterior section to the inferior posterior section.

5. The ankle brace of claim 4, wherein the inferior portion is configured to be disposed inferiorly relative to the sole of the foot of the wearer.

6. The ankle brace of claim 1, wherein the ankle stay is asymmetric over a coronal plane bisecting the ankle of the foot of the wearer.

7. The ankle brace of claim 1, wherein the inferior anterior section comprises a third convex edge facing in the anterior direction and a third concave edge facing in the posterior direction.

8. The ankle brace of claim 1, wherein the main body comprises a medial side configured to be disposed on the medial side of the foot of the wearer and a lateral side configured to be disposed on the lateral side of the foot of the wearer, and the ankle stay is disposed at the medial side of the main body.

9. The ankle brace of claim 1, wherein the main body comprises a medial side configured to be disposed on the medial side of the foot of the wearer and a lateral side configured to be disposed on the lateral side of the foot of the wearer, and the ankle stay is disposed at the lateral side of the main body.

10. The ankle brace of claim 9, wherein the ankle stay is a lateral ankle stay, and further comprising a medial ankle stay coupled to the main body and disposed at the medial side of the main body.

11. An ankle brace comprising:
a main body configured to receive a foot of a wearer;
an ankle stay coupled to the main body, the ankle stay comprising:
an anterior portion configured to be disposed anteriorly relative to the ankle of the foot of the wearer;
an inferior portion coupled to the anterior portion and configured to be disposed inferiorly relative to the sole of the foot of the wearer; and
a posterior portion coupled to the inferior portion opposite the anterior portion, the posterior portion configured to be disposed posteriorly relative to the ankle of the foot of the wearer.

12. The ankle brace of claim 11, wherein the anterior portion comprises a convex edge facing in an anterior direction and a concave edge facing in a posterior direction.

13. The ankle brace of claim 11, wherein the anterior portion comprises a concave edge facing in an anterior direction and a convex edge facing in a posterior direction.

14. The ankle brace of claim 11, wherein the posterior portion comprises a convex edge facing in an anterior direction and a concave edge facing in a posterior direction.

15. The ankle brace of claim 11, wherein the posterior portion comprises a concave edge facing in an anterior direction and a convex edge facing in a posterior direction.

16. The ankle brace of claim 11, wherein the main body comprises a medial side configured to be disposed on the medial side of the foot of the wearer and a lateral side configured to be disposed on the lateral side of the foot of the wearer, and the ankle stay is disposed at the medial side of the main body.

17. The ankle brace of claim 11, wherein the main body comprises a medial side configured to be disposed on the medial side of the foot of the wearer and a lateral side configured to be disposed on the lateral side of the foot of the wearer, and the ankle stay is disposed at the lateral side of the main body.

18. The ankle brace of claim 17, wherein the ankle stay is a lateral ankle stay, and further comprising a medial ankle stay coupled to the main body and disposed at the medial side of the main body.

19. An ankle brace comprising:
a main body configured to receive a foot of a wearer, the main body having a top portion, a bottom portion, a front portion, a rear portion, a first side portion, and a second side portion, the main body comprising:
a first portion disposed between the bottom portion and the top portion and configured to overlay the dorsal surface of the foot of the wearer, the first portion having a first stretchability; and
a second portion disposed at the first side portion and the second side portion, the second portion having a second stretchability, the second stretchability being less than the first stretchability.

20. The ankle brace of claim 19, wherein the second portion is further disposed at the bottom portion of the main body.

21. The ankle brace of claim 19, further comprising an ankle stay disposed at one of the first side portion and the second side portion.

22. The ankle brace of claim 19, further comprising:
a first ankle stay disposed at the first side portion; and
a second ankle stay disposed at the second side portion.

23. An ankle brace comprising:
a main body configured to receive a foot of a wearer, the main body having a first side portion, a second side portion opposite the first side portion, and a sole configured to underlie the foot of the wearer;
a first side stirrup strap coupled to the sole, the first side stirrup strap extending from the sole upwardly and along the first side portion, and the first side stirrup strap detachably coupling to the first side portion; and
a second side stirrup strap coupled to the sole, the second side stirrup strap extending from the sole upwardly and along the second side portion, and the second side stirrup strap detachably coupling to the second side portion.

24. The ankle brace of claim 23, wherein the first side stirrup strap and the second side stirrup strap are monolithically formed with each other.

25. An ankle brace comprising:
a main body configured to receive a foot of a wearer, the main body having a sole configured to underlie the foot of the wearer, a top portion configured to overlie the dorsal surface of the foot of the wearer, and a rear portion configured to be disposed posteriorly relative to the foot of the wearer; and
a first crossing strap and a second crossing strap coupled to the sole, the first crossing strap and the second crossing strap extending in opposite directions from the sole, extending superiorly and crossing each other at the top portion, extending posteriorly and crossing each other again at the rear portion, and extending anteriorly such that the second crossing strap overlies the first crossing strap.

26. The ankle brace of claim 25, wherein the first crossing strap and the second crossing strap are monolithically formed with each other.
